(19) United States
(12) Patent Application Publication
(10) Pub. No.: US 2018/0135122 A1
(43) Pub. Date: May 17, 2018

(54) SYSTEMS AND METHODS FOR GENOTYPE-DERIVED DRUG RECOMMENDATIONS

(71) Applicant: OneOme LLC, Minneapolis, MN (US)

(72) Inventors: Matthew Hilden, Robbinsdale, MN (US); Isabelle Searcy, Plymouth, MN (US); Hennessy Mcllvaine, Minneapolis, MN (US); Matthew Tilstra, Rogers, MN (US); Andy Altepeter, Coon Rapids, MN (US); Jason Sheard, Maple Grove, MN (US)

(21) Appl. No.: 15/349,375

(22) Filed: Nov. 11, 2016

(51) Int. Cl. C12Q 1/68

(52) U.S. Cl. CPC C12Q 1/6876; C12Q 2600/156; C12Q 2600/106

(57) ABSTRACT

A method for providing a tailored drug recommendation for a patient wherein a plurality of metabolizer genes for the patient may be identified from a DNA sample. The metabolizer genes may relate to the patient's ability to metabolize a drug and indicate a metabolizer gene phenotype for the patient. The method may additionally include comparing a drug to the metabolizer gene phenotype and, based on the comparison, assigning a classification to the drug. Moreover, the method may include providing a recommendation for the patient with respect to the drug, the recommendation based at least in part on the assigned classification of the drug. In some embodiments, the method may further include identifying an event/response gene for the patient, the event/response gene related to the patient's compatibility with the drug and indicating an event/response gene phenotype for the patient; and comparing the drug to the event/response gene phenotype.

### FIELD OF THE INVENTION

The present disclosure relates to systems and methods for providing patient-specific drug recommendations for a patient. Particularly, the present disclosure relates to systems and methods for providing drug recommendations for a patient based on the patient's genetic composition. More particularly, the present disclosure relates to systems and methods for evaluating a patient's DNA to determine a genotype and phenotype with respect to one or more drugs.

### BACKGROUND OF THE INVENTION

The background description provided herein is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

Patients receive prescription and non-prescription medications for treating various medical conditions, such as hypertension, high cholesterol, hypothyroidism, diabetes, pain management, heartburn, epilepsy, cancer, rheumatoid arthritis, mood and sleep disorders, and other medical conditions. Billions of prescription medications are filled each year in the United States. A prescription or non-prescription drug can have a varied response in different patients. In some cases, patients may experience adverse responses, including side effects, ineffective dosing, and/or other problems with a medication. A patient may try multiple prescription and/or non-prescription medications for treating a particular condition until the patient finds a particular medication that they respond well to, providing effectiveness with little to no side effects. Some adverse reactions to prescription or non-prescription medications can detrimentally affect a patient's health.

The varied response to prescription medications is due, at least in part, to patients' genetic factors. For example, while some patients may be genetically predisposed to process a particular drug relatively well, other patients' genetic makeup may make the drug's metabolization slower, or much faster than normal. Another example is that some patients may be genetically predisposed to develop a side effect with a particular medication, while others will not have this predisposition. In many cases, a prescribing doctor or other medical professional may not be aware of the patient's particular genotype or phenotype with respect to particular drugs. In this way, the provider lacks critical information that can help in identifying a drug that best treats the patient for a particular condition and minimize trial and error to determine which drug operates best with the patient's genotype.

Thus, there is a need in the art for systems, and methods for genotype-derived recommendations for medication use.

### BRIEF SUMMARY OF THE INVENTION

The following presents a simplified summary of one or more embodiments of the present disclosure in order to provide a basic understanding of such embodiments. This summary is not an extensive overview of all contemplated embodiments, and is intended to neither identify key or critical elements of all embodiments, nor delineate the scope of any or all embodiments.

The present disclosure, in one or more embodiments, relates to a method for providing a tailored drug recommendation for a patient. The method may include receiving a DNA sample for the patient. The method may further include identifying a plurality of metabolizer genes for the patient, the metabolizer genes related to the patient's ability to metabolize a drug and indicating a metabolizer gene phenotype for the patient. The method may additionally include comparing a drug to the metabolizer gene phenotype and, based on the comparison, assigning a classification to the drug. Moreover, the method may include providing a recommendation for the patient with respect to the drug, the recommendation based at least in part on the assigned classification of the drug. In some embodiments, the classification may be a metabolizer classification. In some embodiments, the method may further include identifying an event/response gene for the patient, the event/response gene related to the patient's compatibility with the drug and indicating an event/response gene phenotype for the patient, and comparing the drug to the event/response gene phenotype. Moreover, the method may further include, based on the comparison of the drug to the event/response gene phenotype, assigning an event/response classification to the drug; and assigning an overall drug classification to the drug, the overall drug classification based on the metabolizer classification and the event/response classification. In some embodiments, comparing a drug to the metabolizer gene phenotype may include calculating a metabolizer score for each metabolizer gene, identifying one or more of the plurality of metabolizer genes capable of metabolizing the drug, assigning a weight to the one or more metabolizer genes capable of metabolizing the drug, multiplying the metabolizer score for each metabolizer gene capable of metabolizing the drug by its assigned weight to obtain a weighted metabolizer score, and summing the weighted metabolizer scores to obtain a drug score for the drug. In some embodiments, assigning a classification to the drug may include comparing the drug score to a plurality of classifications. In some embodiments, the metabolizer classification, event/response classification, and/or overall drug classification may be one of: use as directed, use with caution, and use with great caution. In some embodiments, the overall drug classification may be either the metabolizer classification or the event/response classification. Further, the metabolizer phenotype may be poor metabolizer, poor to intermediate metabolizer, intermediate metabolizer, intermediate to normal metabolizer, normal metabolizer, normal to ultrarapid metabolizer, ultrarapid metabolizer, intermediate to ultrarapid metabolizer, poor to normal metabolizer, or poor to ultrarapid metabolizer. In some embodiments, providing a recommendation may include providing a report. The recommendation may be provided to a medical professional or a patient. In some embodiments, providing a recommendation may include providing an interactive recommendations terminal. Identifying a plurality of metabolizer genes for the patient may include identifying the alleles for each of the metabolizer genes. Moreover, calculating a metabolizer score for each metabolizer gene may include, for each metabolizer gene, identifying the alleles for the gene, assigning an allele score to each allele, and summing the allele scores to obtain a metabolizer score.

The present disclosure, in one or more additional embodiments, relates to a method for providing a tailored drug recommendation for a patient. The method may include receiving DNA data for the patient and identifying a genotype for the patient with respect to a metabolizer gene related to the patient's ability to metabolize a drug and/or an event/response gene related to the patient's compatibility with the drug. Further, the method may include identifying a phenotype for the patient with respect to the metabolizer gene and/or event/response gene, and providing a recommendation for the patient with respect to the drug. In some embodiments, providing a recommendation may include comparing the drug to the phenotype.

The present disclosure, in one or more additional embodiments, relates to a system for providing a tailored drug recommendation for a patient. The system may include a database storing data related to one or more drug-gene relationships, and a controller configured to provide a tailored drug recommendation. In some embodiments, the controller may have a patient evaluation module, a drug evaluation module, and a recommendations module. The patient evaluation module may be configured to evaluate DNA data for the patient to identify one or more genes related to the patient's ability to metabolize a drug. The drug evaluation module may be configured to compare the one or more identified genes with the drug. Moreover, the recommendations module may be configured to provide one or more tailored drug recommendations for the patient. In some embodiments, the system may further have a user interface providing the one or more tailored drug recommendations through an interactive recommendations terminal.

While multiple embodiments are disclosed, still other embodiments of the present disclosure will become apparent to those skilled in the art from the following detailed description, which shows and describes illustrative embodiments of the invention. As will be realized, the various embodiments of the present disclosure are capable of modifications in various obvious aspects, all without departing from the spirit and scope of the present disclosure. Accordingly, the drawings and detailed description are to be regarded as illustrative in nature and not restrictive.

### BRIEF DESCRIPTION OF THE DRAWINGS

While the specification concludes with claims particularly pointing out and distinctly claiming the subject matter that is regarded as forming the various embodiments of the present disclosure, it is believed that the invention will be better understood from the following description taken in conjunction with the accompanying Figures, in which:

FIG. 1 is a flowchart of a method of the present disclosure, according to one or more embodiments.

FIG. 2 is a diagram of a system of the present disclosure, according to one or more embodiments.

FIG. 3 is a flowchart of a process of the present disclosure, according to one or more embodiments.

FIG. 4A is an example page of a patient report of the present disclosure, according to one or more embodiments.

FIG. 4B is another example page of a patient report of the present disclosure, according to one or more embodiments.

FIG. 4C is another example page of a patient report of the present disclosure, according to one or more embodiments.

FIG. 4D is another example page of a patient report of the present disclosure, according to one or more embodiments.

FIG. 4E is another example page of a patient report of the present disclosure, according to one or more embodiments.

FIG. 4F is another example page of a patient report of the present disclosure, according to one or more embodiments.

FIG. 4G is another example page of a patient report of the present disclosure, according to one or more embodiments.

FIG. 4H is another example page of a patient report of the present disclosure, according to one or more embodiments.

FIG. 4I is another example page of a patient report of the present disclosure, according to one or more embodiments.

FIG. 4J is another example page of a patient report of the present disclosure, according to one or more embodiments.

FIG. 4K is another example page of a patient report of the present disclosure, according to one or more embodiments.

FIG. 5A is an example of an interactive reporting terminal of the present disclosure, according to one or more embodiments.

FIG. 5B is another example of an interactive reporting terminal of the present disclosure, according to one or more embodiments.

FIG. 5C is another example of an interactive reporting terminal of the present disclosure, according to one or more embodiments.

FIG. 5D is another example of an interactive reporting terminal of the present disclosure, according to one or more embodiments.

FIG. 5E is another example of an interactive reporting terminal of the present disclosure, according to one or more embodiments.

FIG. 5F is another example of an interactive reporting terminal of the present disclosure, according to one or more embodiments.

FIG. 5G is another example of an interactive reporting terminal of the present disclosure, according to one or more embodiments.

FIG. 5H is another example of an interactive reporting terminal of the present disclosure, according to one or more embodiments.

FIG. 5I is another example of an interactive reporting terminal of the present disclosure, according to one or more embodiments.

FIG. 5J is another example of an interactive reporting terminal of the present disclosure, according to one or more embodiments.

FIG. 6 is an example of a process of the present disclosure for resolving potential allele ambiguities in genes having heterozygous mutation, according to one or more embodiments.

### DETAILED DESCRIPTION

The present disclosure, in one or more embodiments, relates to novel and advantageous systems and methods for providing patient-specific recommendations for one or more drugs, medications, or other substances (referred to herein collectively as “drugs” or “medications”). Systems and methods of the present disclosure may include, but are not limited to, knowledge curation, patient genotype evaluation, drug evaluation, and one or more recommendations. Knowledge curation may include, but is not limited to, mining one or more sources for data related to drug-gene relationships and evaluating the data with respect to evidence criteria. Patient genotype evaluation may include, but is not limited to, receiving a patient DNA sample, identifying one or more metabolizer genes and/or event/response genes from the sample, and identifying one or more resulting phenotypes. Drug evaluation may include, but is not limited to, comparing one or more drugs to the patient genotype evaluation based on the one or more drug-gene relationships identified by knowledge curation. Specifically, drug evaluation may include, but is not limited to, determining a drug score for one or more drugs, classifying the one or more drugs with respect to one or more metabolizer genes and/or one or more event/response genes, and determining one or more clinical annotations. Further, one or more recommendations may be provided for the patient. The one or more recommendations may relate to the patient's compatibility with or ability to metabolize the one or more drugs. The one or more recommendations may be provided in a report and/or via an interactive terminal in some embodiments.

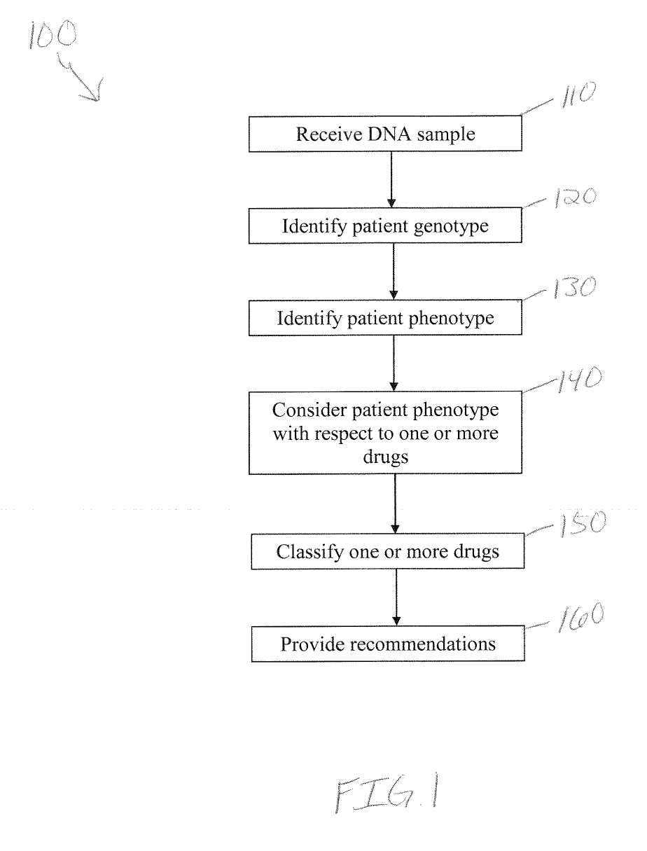

Turning now to FIG. 1, a method 100 of providing a genotype derived medication recommendation, according to one or more embodiments, is shown. The method 100 may include receiving a DNA sample 110; identifying a patient genotype 120; identifying a patient phenotype 130; considering the patient phenotype with respect to one or more drugs 140; classifying the one or more drugs 150; and providing one or more recommendations 160.

The method 100 may include receiving a patient's DNA sample 110. The DNA sample may be received in any suitable form. For example, the DNA sample may be received as, or derived from, a cheek swab, a blood draw, another bodily fluid sample, a hair sample, or any other suitable patient DNA source. The sample may be received from a physician, laboratory, hospital, or other medical professional or establishment. In some embodiments, the sample may be received from the patient. In still other embodiments, the sample may be received from any other suitable source. In other embodiments, DNA data derived from a patient's DNA may be received without the DNA sample itself. That is, for example, data related to the patient's genes, including allele combinations, may be received in any suitable form.

The method 100 may further include identifying a patient genotype 120 from the received DNA sample. That is, the DNA sample may be analyzed to identify one or more genes of the patient. The one or more genes may be further analyzed to determine the patient's allele pairs. The one or more genes analyzed may include metabolizer genes. Metabolizer genes may generally contribute to or have an effect on the patient's metabolization of medications or other substances. The one or more genes analyzed may additionally or alternatively include one or more event/response genes. An event/response gene may be a gene identified as having an effect on the patient's metabolism of, processing of, or response to a particular drug in some embodiments. An event/response gene may be a gene identified as relating to a patient's potential for an adverse reaction to a particular drug in some embodiments. For example, the presence or absence of a particular allele combination for a particular event/response gene may indicate the likelihood of side effects with respect to a particular drug. In other embodiments, as described above, the genotype data may be received without a DNA sample.

The method 100 may further include identifying a patient phenotype 130 in some embodiments. In some embodiments, the patient phenotype may be identified based on the identified genotype. A patient phenotype may include one or more expected physical or metabolic responses or attributes that the patient may exhibit as a result of the patient's genotype. For example, the patient phenotype may include an expected metabolic rate for metabolizing one or more drugs as a result of the patient's particular metabolizer genes. Moreover, the patient phenotype may include one or more expected responses as a result of the one or more identified event/response genes. For example, where the presence of a particular event/response gene allele combination indicates a risk for side effects with respect to a particular drug, and the patient's genotype indicates the presence of that allele combination, the patient's phenotype may include a risk for such side effects. As an example, some gene allele combinations may indicate a patient's sensitivity level to the anticoagulant drug, warfarin, which can in turn indicate a risk for possible side effects. Based on the particular alleles identified for the patient with respect to step 120, the patient's phenotype may include a low, intermediate, or high sensitivity to warfarin.

The method 100 may include considering the patient's identified phenotype and/or genotype with respect to one or more drugs 140. That is, one or more drugs or medications may be compared to the patient's expected physical or metabolic responses or attributes identified in step 130 and/or the patient's identified genetic data. This comparison may determine, for example, whether the one or more drugs are suitable for the patient.

Moreover, the method 100 may include classifying the one or more drugs 150. That is, one or more drugs may be classified based on the consideration step 140 with respect to the patient's phenotype and the one or more drugs. The drug classification may generally indicate the drug's suitability for, or compatibility with, the patient's DNA. For example, in some embodiments, drug classifications may signify one or more drugs that the patient may take as directed, one or more drugs that the patient may take with caution, and one or more drugs that the patient may take with great caution. In some embodiments, drug classifications may be color coded. For example, a drug may be classified as red where the drug should be taken with great caution, based on comparison to the patient's phenotype and/or genotype. A drug may be classified as yellow where the drug should be taken with caution, based on comparison to the patient's genotype and/or phenotype. Further, a drug may be classified as green where the drug may be taken as directed, based on comparison to the patient's genotype and/or phenotype. In other embodiments, drug classifications may signify any other suitable groupings for one or more drugs and may be designated by numbers, letters, and/or any other suitable identifiers.

In some embodiments, the method 100 may include providing one or more recommendations 160. That is, the result of the consideration step 140 may be presented as one or more recommendations. The one or more recommendations may include the drug classifications of step 150 in some embodiments. That is, the one or more recommendations may include, for example, a list of drugs that the patient may take as directed, should take with caution, and/or should take with great caution. The one or more recommendations may be provided in a report in some embodiments, such as an electronic or physical report. Additionally or alternatively, the one or more recommendations may be provided via an interactive user terminal. The one or more recommendations may be provided to a healthcare professional in some embodiments, such as a doctor, pharmacist, laboratory technician, or other suitable healthcare professional. Additionally or alternatively, the one or more recommendations may be provided to the patient.

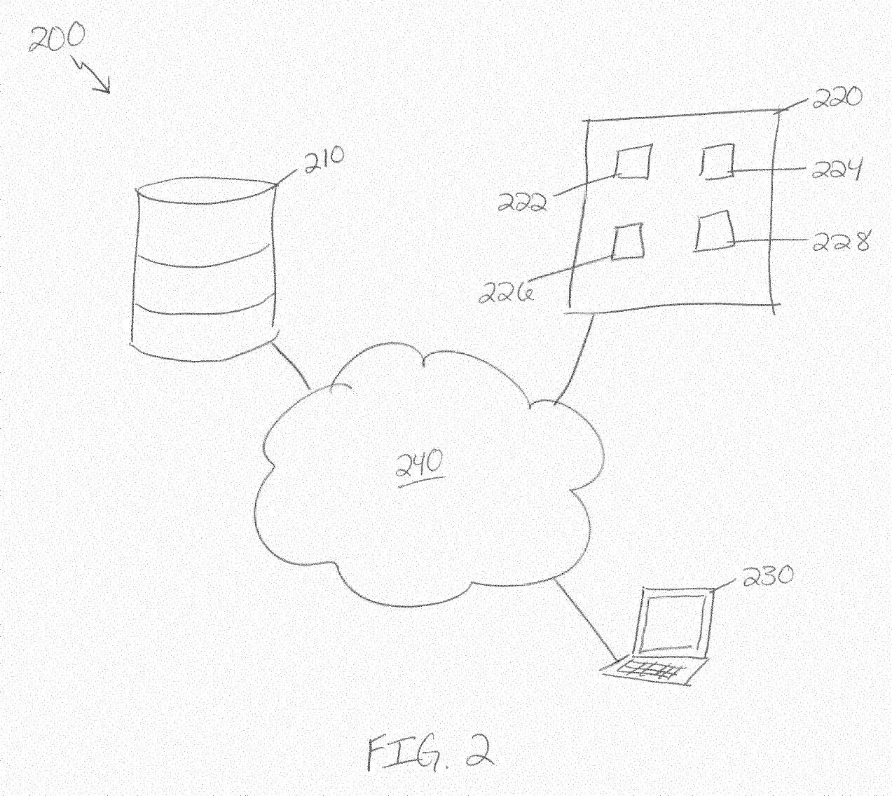

A method or process of the present disclosure may be performable by a system in some embodiments. FIG. 2 shows a system 200 according to one or more embodiments that may be configured to perform a process or method of the present disclosure. As shown in FIG. 2, the system 200 may include a database 210, a controller 220, and a user interface 230 communicably coupled via a network 240. The network 240 may generally be a wired or wireless network. In some embodiments, the network may be an Internet network, cloud network, and/or any other suitable network.

The database 210 may generally include one or more types of computer readable storage media, such as one or more solid state drives, flash drives, and/or any other suitable storage type(s). In some embodiments, the database 210 may store data related to one or more drugs. For example, the database 210 may store data related to how one or more drugs is metabolized, data related to possibility or probability of side experiencing side effects from one or more drugs, genetic markers for drug metabolization and/or side effects, drug interactions, and/or other drug data. Additionally or alternatively, the database 210 may store data related to one or more genes. For example, the database 210 may store phenotype data for one or more gene combinations and/or allele combinations. That is, the database 210 may store one or more expected or documented phenotypes consistent with one or more gene or allele combinations. In some embodiments, the database 210 may store data related to one or more drug-gene relationships. Particularly, the database 210 may store data related to the ability of a patient to metabolize or otherwise process or respond to a particular drug based on the presence or absence of one or more allele combinations or other genetic markers. In some embodiments, the database 210 may store additional or alternative information.

The controller 220 may have hardware and/or software for curating and evaluating drug and gene data, analyzing a patient's DNA sample to identify a genotype and phenotype, analyzing one or more drugs in connection with the patient's genotype or phenotype, and/or providing one or more patient recommendations. In some embodiments, the controller 220 may have a knowledge curation module 222, a patient evaluation module 224, drug evaluation module 226, and a recommendations module 228. In other embodiments, the controller 220 may have additional or alternative modules or elements.

The knowledge curation module 222 may include hardware and/or software for collecting and/or evaluating drug data and/or gene data to be included in the database 210. In some embodiments, the knowledge curation module 222 may access one or more sources to find or gather data related to one or more drugs, genes, or drug-gene relationships. For example, the knowledge curation module 222 may search one or more public or private databases, published or unpublished studies, research papers, articles, data provided by one or more government agencies, other literature; and/or other suitable public or private sources. The data gathered may relate to one or more drugs, one or more genes, and/or one or more drug-gene relationships. In some embodiments, the knowledge curation module 222 may analyze the data related to one or more drugs and/or one or more genes to develop one or more drug-gene relationships. A drug-gene relationship may provide an indication, for example, on how a patient having a particular gene or particular allele or allele combination may respond to a particular drug. A drug-gene relationship may relate to one or more metabolizer genes and/or one or more event/response genes, for example. The knowledge curation module 222 may develop drug-gene relationships based on data gathered from one or more sources.

In some embodiments, the knowledge curation module 222 may additionally evaluate the data against one or more evidence criteria to determine whether the data should be used in evaluating drugs, genes, determining drug-gene relationships, and/or making medication recommendations. That is, the knowledge curation module 222 may evaluate the data gathered from one or more sources for its relevance, accuracy, and/or other quality factors to determine whether the data should be used in evaluating drugs, genes, determining drug-gene relationships, and/or making medication recommendations. Evidence criteria for evaluating whether data should be used may consider the data's source, the usefulness of the data, the prevalence of the data, and/or other qualities such as, for example, the size of study where the data relates to a medical study. In some embodiments, the evidence criteria may include a scale, such as a numerical scale, indicating differing levels of data quality. Data meeting particular desired level(s) of quality on the scale may be used for determining one or more drug-gene relationships, classifying one or more drugs or genes, and/or providing one or more medication recommendations. In some embodiments, data not meeting the desired level(s) of quality may be, for example, annotated with an indication that the data may not meet a particular desired level of quality for use. In other embodiments, data not meeting the desired level(s) may be excluded from the database 210. In some embodiments, the knowledge curation module 222 may search for and/or evaluate data from one or more sources continuously or intermittently. In other embodiments, the knowledge curation module 222 may search for and/or evaluate data from one or more sources on demand.

The patient evaluation module 224 may generally evaluate a patient's genetic makeup. For example, in some embodiments, the patient evaluation module 224 may analyze a DNA sample received for a patient. The patient evaluation module 224 may identify one or more genes and, in some embodiments, one or more alleles for the identified genes. In some embodiments, the patient evaluation module 224 may evaluate the patient DNA for one or more metabolizer genes and/or one or more event/response genes. Particularly, in some embodiments, the patient DNA may be evaluated with respect to the one or more metabolizer genes and/or event/response genes identified by the knowledge curation module 222 as part of a drug-gene relationship. Moreover, in some embodiments, the patient evaluation module 224 may identify one or more patient phenotypes based on the patient's gene data. That is, the patient evaluation module 224 may identify one or more expected physical, metabolic, or other traits or responses that a patient may exhibit in response to one or more drugs based on drug and/or gene data in the database 210.

The drug evaluation module 226 may evaluate one or more drugs identified by the knowledge curation module 222 with respect to the patient's genotype(s) and/or phenotype(s) identified by the patient evaluation module 224. That is, for example, the drug evaluation module 226 may compare one or more drugs in the database 210 to the patient's genetic data to determine whether each drug may be compatible with, or appropriate for, the patient. Such comparison may be based on the patient's one or more identified metabolizer genes and/or event/response genes. For example, where a drug-gene relationship identified by the knowledge curation module 222 indicates that a patient having a particular allele combination for a gene may have a relatively low tolerance for a particular drug, the drug evaluation module 226 may identify this drug as sub-optimal or inappropriate for the patient. In some embodiments, the drug evaluation module 226 may evaluate each drug identified in the database 210. Moreover, in some embodiments, the drug evaluation module 226 may categorize the one or more drugs based on its effectiveness, compatibility, suitability, and/or other factors with respect to the patient's DNA.

The recommendations module 228 may provide one or more drug recommendations based on the analyses performed by the patient evaluation module 224 and the drug evaluation module 226. For example, the recommendations module 228 may provide a recommendation for the one or more drugs in the database 210. That is, the recommendations module 228 may provide guidance as to medications that may be generally compatible or incompatible with the patient's genotype. For example, where analysis determined that a drug was incompatible with or otherwise unsuitable for the patient, the recommendations module 228 may recommend that the patient use the drug with great caution and/or may provide a clinical annotation informing the provider that an alternative may be more suited. The recommendations module 228 may recommend that the patient should take some drugs with varying levels of caution and/or may take other drugs as directed and/or may adjust the dose. In some embodiments, the recommendations module 228 may provide specific recommendations with respect to one or more drugs, such as suggesting that particular dosages or frequencies be used by the patient. The one or more recommendations may be stored in the database 210 in some embodiments. A drug recommendation may be provided to the patient whose DNA was analyzed by the system 200, or to a doctor, nurse, pharmacist, laboratory technician, and/or other medical professional. Moreover, a drug recommendation may be provided in any suitable form. For example, in some embodiments, one or more drug recommendations may be provided as a digital or physical report. In other embodiments, one or more drug recommendations may be provided via the user interface 230.

With continued reference to FIG. 2, the user interface 230 may include one or more software programs or applications for accessing one or more recommendations provided by the recommendations module 228 and/or other data stored in the database 210. The user interface 230 may be accessible via a desktop computer, laptop computer, tablet computer, smartphone, smart watch, and/or any other suitable user device. In some embodiments, the user interface 230 may provide an interactive terminal for a user to access the one or more recommendations and/or other data. For example, a doctor, nurse, or other medical professional may access the user interface 230 to determine a medication to prescribe to the patient. Additionally or alternatively, the patient may access the user interface 230 to view the one or more recommendations and determine the patient's compatibility with one or more drugs. In some embodiments, the user interface 230 may be an interactive terminal providing various navigable screens, pages, and/or other components.

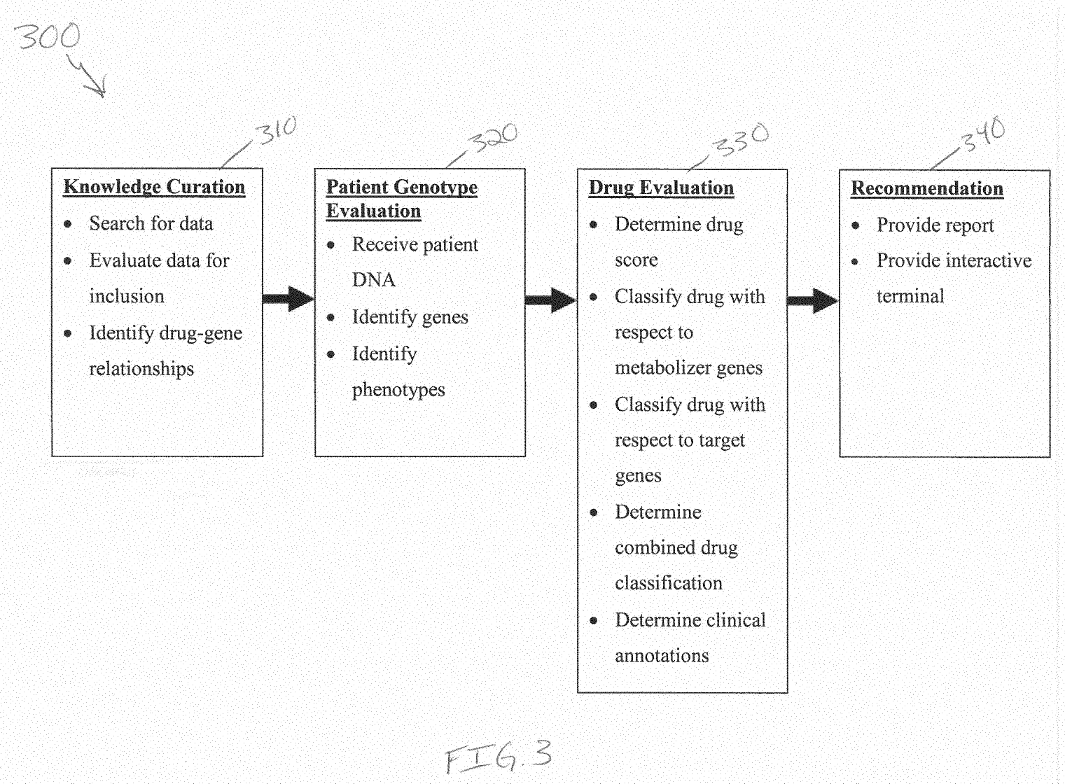

Turning now to FIG. 3, a process 300 for providing one or more drug recommendations for a patient is shown. As shown, the process 300 may include knowledge curation 310, patient genotype evaluation 320, drug evaluation 330, and recommendation 340. In other embodiments, the process 300 may include additional or alternative components than those shown in FIG. 3.

Knowledge curation 310 may generally include compiling, evaluating, and/or developing data related to one or, more drugs, genes, and/or drug-gene relationships. In some embodiments, curating knowledge 310 may include building a database of knowledge related to drug-gene relationships, such that a patient's response to one or more drugs may be predicted based on genetic factors. Knowledge curation 310 may include searching for data related to the one or more drugs, genes, or drug-gene relationships, evaluating the data for quality, and identifying one or more drug-gene relationships supported by the data.

Knowledge curation 310 may include searching a variety of sources. For example, drug and/or gene data may be derived from the United States Food and Drug Administration (FDA), including FDA drug labels, the FDA Table of Pharmacogenomic Biomarkers in Drug Labels, and/or other FDA sources; the Mayo Clinic and/or other research institutions; published or unpublished studies, research papers, articles, or other literature; and/or other suitable public or private sources. In some embodiments, one or more public or private data sources, such as for example third party pharmacogenomics databases, medical publication databases, and Internet search engines may be used to search for or gather drug and/or gene data. Some databases that may be used to search for or gather data may include, but are not limited to, Clinical Pharmacogenetics Implementation Consortium (CPIC), Pharmacogenomics Knowledge Base, Lexicomp, PubMed, Transformers (formerly SuperCYP), OMIM, and DrugBank.

Knowledge curation 310 may search for data such as, but not limited to, for a particular drug, the generic drug name; drug class and sub-class; metabolizing cytochromes (CYP) for the drug, including the degree to which each cytochrome contributes to the metabolization of the drug (such as whether the CYP is a “major” or “minor” contributor, for example); event/response genes that may contribute to or affect the pharmacokinetics, pharmacodynamics, effectiveness, or use of the drug; drug activity (whether active or inactive compound); and/or metabolites activity (whether active or inactive drug metabolites). Other data sought or gathered for one or more drugs may include dosing guidelines, information on genomic biomarkers, provider considerations, and/or other data relating to prescribing or using the drug. Moreover, for one or more gene combinations or allele combinations, knowledge curation 310 may include searching for phenotypes. That is, for a particular gene combination or allele combination, expected physical or metabolic traits or responses to one or more drugs may be sought. Other data related to one or more drugs, genes, or drug-gene relationships may be additionally or alternatively sought or gathered in some embodiments.

Knowledge curation 310 may additionally include evaluating the gene and/or drug data gathered from one or more sources for its relevance, accuracy, quality, and/or other factors to determine whether the data should be used for evaluating drugs, genes, determining drug-gene relationships, or providing recommendations based on the patient's genotype and/or phenotype. In some embodiments, the drug and/or gene data may be evaluated with respect to evidence criteria configured to distinguish data that should be used for the listed purposes from data that may be otherwise unsuitable for use (i.e. data with enough evidence versus data with insufficient evidence). Evidence criteria for determining whether data should be used may evaluate the data based on its source, the type of data, amount of data, and/or other factors. In some embodiments, evidence criteria may be arranged on a scale, such as a numeric scale, for categorizing data. The evidence criteria scale may generally represent varying degrees of data quality.

For example, in some embodiments, the evidence criteria scale against which data may be analyzed to determine whether it should be used for medication recommendations may have five levels: 1A, 1B, 2A, 2B, and 3 representing varying levels of data quality. Level 1A may represent a highest level of data quality, whereas level 3 may represent a lowest level of data quality. For example, data that corresponds with Level 1A on the evidence criteria scale may be particularly useful or supported data, such as specific drug dosing guidance provided by the FDA or another public or well-regarded institution. Data that corresponds with Level 1B on the evidence criteria scale may be at least slightly less relevant or comprehensive than Level 1A data. Data that corresponds with Level 1B may include warnings by the FDA or another public or well-regarded institution without specific drug dosing guidance. Other Level 1B data may include a variant-drug combination where the data shows a relatively strong association replicated in more than one study with significant p-values and a strong effect size. Data that corresponds with Level 2A may include less comprehensive data provided by the FDA or another public or well-regarded institution, such as a statement that a drug is metabolized or interacts with a particular gene. Data that corresponds with Level 2B may include an annotation for a variant-drug combination with moderate evidence of a replicated association. Data that corresponds with Level 3 may include, for example, an annotation for a variant-drug combination based on a single significant but not yet replicated study, or an annotation for a variant-drug combination evaluated in multiple studies but lacking clear evidence of an association. In some embodiments, only data meeting criteria Levels 1A, 1B, 2A, or 2B may be used for providing medication recommendations. In other embodiments, only data meeting Levels 1A or 1B may be used for providing medication recommendations. In still other embodiments, other levels of data may be used for providing medication recommendations. Moreover, in other embodiments, a data evidence criteria scale may include additional or alternative categories of data quality, each of which may be represented by any suitable number, letter, color, or other identifier.

Knowledge curation 310 may additionally include identifying or determining one or more drug-gene relationships such that a patient's DNA may be evaluated with respect to one or more drugs. In some embodiments, one or more drug-gene relationships may be expressly or clearly identified in the curated data. In other embodiments, knowledge curation 310 may include extrapolating the one or more drug-gene relationships from the curated drug and/or gene data. For example, knowledge curation 310 may include drawing conclusions as to the particular effects that an individual gene or allele combination may have with respect to a patient's use of a particular drug based on curated data in some embodiments.

With continued reference to FIG. 3, the process 300 may include patient genotype evaluation 320, which may evaluate the DNA of one or more patients to determine the presence or absence of particular genes, alleles, mutations, and/or other genetic components that may be relevant to the patient's potential for metabolizing one or more drugs. Patient genotype evaluation may include receiving a patient DNA sample, identifying one or more genes, and identifying one or more phenotypes.

In some embodiments, patient genotype evaluation 320 may include receiving a DNA sample. As described above with respect to the method 100, the DNA sample may be received in any suitable form. For example, the DNA sample may be received as, or derived from, a cheek swab, a blood draw, or any other suitable patient DNA source. The sample may be received from a physician, nurse, laboratory, hospital, or other medical professional or establishment. In some embodiments, the sample may be received from the patient. In still other embodiments, the sample may be received from any other suitable source.

The received DNA sample may be evaluated to identify one or more patient genes. Identifying one or more patient genes from the DNA sample may include identifying one or metabolizer genes in some embodiments. Metabolizer genes may be genes related to or involved in the general metabolism of drugs. For example, some metabolizer genes may be cytochromes such as CYP1A2, CYP2B6, CYP2C9, CYP2C19, CYP2D6, CYP3A4, CYP3A5, and/or any other suitable genes. Any suitable number of metabolizer genes may be identified from the patient DNA sample. In some embodiments, for example, between 1 and 10 metabolizer genes may be identified. Particularly, in some embodiments, between 6 and 8 metabolizer genes may be identified. In other embodiments, any suitable number of metabolizer genes may be identified. The metabolizer genes identified may be identified due to their presence in a drug-gene relationship identified by knowledge curation 310 in some embodiments.

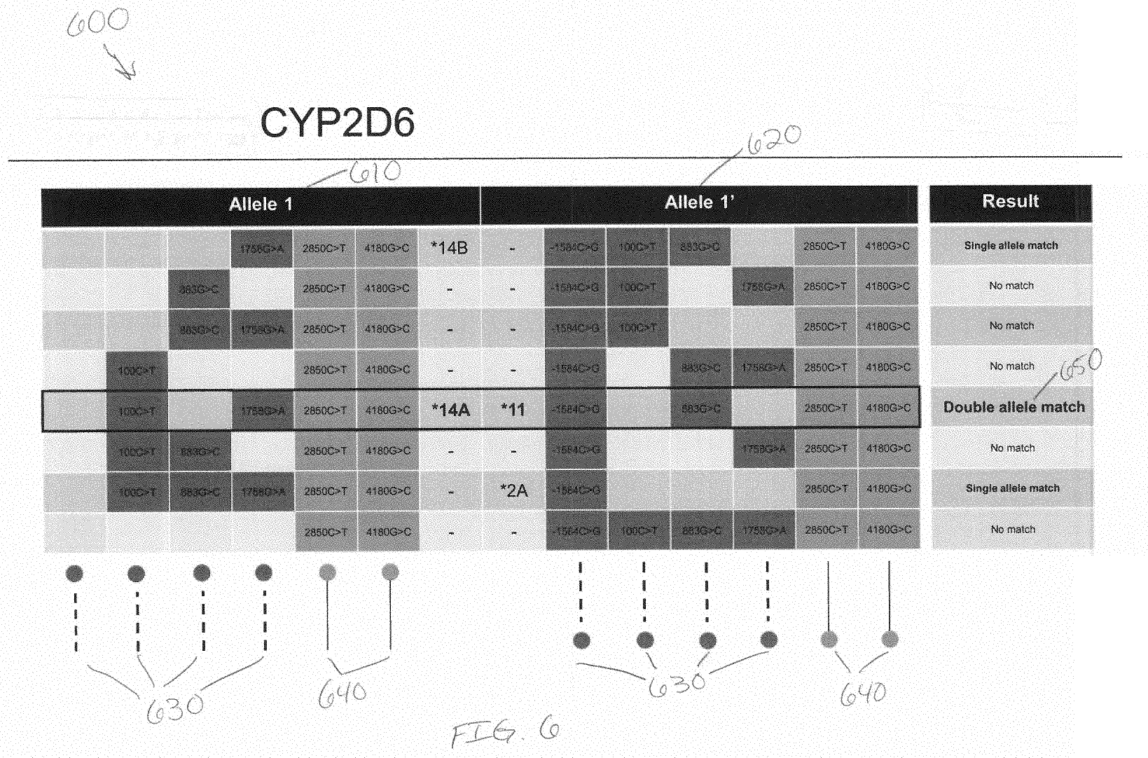

In some embodiments, identification of metabolizer genes may include resolving potential allele source ambiguities in genes having heterozygous mutations. An allele source ambiguity may exist where, for example, the two alleles identified for a particular gene may be indeterminate from a patient's DNA sample due to heterozygous mutations on one or more single-nucleotide polymorphism (SNP) locations. An inability to determine whether one or more SNP mutations are maternal or paternal may affect the ability to determine the patient's allele combination with respect to the ambiguous genotype. Thus, in some embodiments, the source ambiguity may be resolved by comparing all possible or likely permutations of allele combinations the patient may have for the gene. That is, for each allele of the gene, all possible SNP permutations may be examined. The possible or likely SNP permutations may be available based on the curated knowledge. Further, based on known SNPs for the patient, such as homozygous SNPs, the matching alleles may be selected from the possible permutations. FIG. 6 illustrates an example of a permutation calculation 600 for a first allele 610 and a second allele 620 for a gene. As shown, all possible or likely heterozygous SNPs 630 may be examined in combination with identified homozygous variant SNPs 640. These permutations may be matched against published allele combinations (i.e. allele combinations identified based on curated knowledge) to signify the patient's likely haplotype.

In addition to, or alternative to, identifying one or more metabolizer genes, one or more event/response genes may be identified for the patient in some embodiments. Event/response genes may be genes that relate generally to pharmacodynamics, bioavailability of, excretion of, or adverse reactions to one or more drugs. For example, some event/response genes may be COMT, DPYD, DRD2, F2, F5, GRIK4, HTR2A, HTR2C, IL28B (IFNL3), NUDT15, OPRM1, SLCO1B1, TPMT, UGT1A1, VKORC1, and/or any other suitable genes. Identifying the one or more event/response genes may include determining whether the patient has the one or more genes and/or whether the patient has particular alleles associated with the gene(s). For example, with respect to UGT1A1, it may be determined whether the patient has one or two functioning or deficient alleles with respect to the gene. Two deficient UGT1A1 alleles may indicate a high risk for hyperbilirubinemia, toxicity, and/or other adverse effects with respect to one or more drugs. One deficient allele with one functioning allele may indicate an increased risk, and two functioning alleles may indicate a normal risk. The event/response genes identified may be identified due to their presence in a drug-gene relationship identified by knowledge curation 310 in some embodiments.

In some embodiments, one or more identified metabolizer and/or event/response genes may be converted into a standard identification format. For example, in some embodiments, a star allele nomenclature may be used for identifying the one or more metabolizer genes and/or event/response genes. In other embodiments, other suitable naming or identification formats may be used. Conversion to star allele or any other suitable format may allow the various metabolizer, event/response, and/or other genes to be more easily referenced in patient reports and/or other products.

Patient genotype evaluation 320 may additionally include identifying one or more phenotypes in some embodiments. A phenotype may be an observable trait, such as a biochemical or physiological property resulting from a particular gene. To determine a phenotype for a particular gene, both alleles of the gene may be examined. In some embodiments, a metabolizer phenotype may be identified with respect to one or more metabolizer genes, and/or an event/response phenotype may be identified with respect to one or more event/response genes.

For example, in some embodiments, a metabolizer phenotype may be identified for each of the one or more identified metabolizer genes. Possible phenotypes for metabolizer genes may include, but are not limited to, poor metabolizer, poor to intermediate metabolizer, intermediate metabolizer, intermediate to normal metabolizer, normal metabolizer, normal to ultrarapid metabolizer, ultrarapid metabolizer, intermediate to ultrarapid metabolizer, poor to normal metabolizer, and poor to ultrarapid metabolizer. In some embodiments, a metabolizer phenotype may be determined for a metabolizer gene by assigning a metabolizer gene score, such a numerical score, to the gene. The metabolizer gene score for a metabolizer gene may be determined based on the curated knowledge. In some embodiments, a metabolizer gene score for a metabolizer gene may be calculated by examining the alleles for the gene and assigning an individual allele score for each allele. The particular numerical scores assigned to each allele may be based on the curated knowledge. In some embodiments, allele scores may be determined based on the expected protein activity for a particular allele. For example, where an allele is expected to yield a protein with no or low activity with respect to metabolizing drugs, the allele may be assigned a relatively low allele score. However, where an allele is expected to have an increased protein activity with respect to metabolizing drugs, the allele may be assigned a relatively high allele score. Moreover, an allele expected to have a normal or average protein activity may be assigned a moderate allele score. In some embodiments, the range of assignable allele scores may be normalized to an average protein activity or moderate protein activity observed in a plurality of patients carrying the same alleles. The assignable allele scores may range between approximately zero and approximately 10 in some embodiments. Particularly, the assignable allele scores may range between approximately 0 to approximately 5 in some embodiments. In other embodiments, the assignable allele scores may have any other suitable range based on curated knowledge.

The allele scores for a metabolizer gene may be combined to reach a metabolizer gene score in some embodiments. For example, the allele scores for two alleles of a metabolizer gene may be summed to reach a metabolizer gene score signifying the expected metabolic phenotype of the gene. Examples of metabolizer gene score calculations for three metabolizer genes based on assigned allele scores are shown below:

CYP2D6
Star allele combination: *2A/*35B
Assigned allele scores: 1.4/1.0
Metabolizer score: 1.4+1.0=2.4

CYP3A5
Star allele combination: *1/*22
Allele scores: 1.0/0.5
Metabolizer score: 1.0+0.5=1.5

CYP3A5
Star allele combination: *1/*1
Assigned allele scores: 1.6/1.6
Metabolizer score: 1.6+1.6=3.2

Additionally or alternatively, an event/response phenotype may be identified for each of the one or more identified event/response genes. For example, based on the particular alleles present, some event/response genes may have a tendency to cause one or more effects with respect to one or more drugs when used by a patient. Each of the above identified event/response genes for the patient may be evaluated to determine which allele combinations are present. The patient's phenotype with respect to each event/response gene may then be identified. For example, as described above, the event/response gene UGT1A1 may affect a patient's risk for hyperbilirubinemia and toxicity with respect to metabolization of particular drugs. The event/response gene may be examined to determine whether the patient has one or two functioning or deficient alleles. If two functioning alleles are identified, the patient's phenotype with respect to UGT1A1 may be identified as normal risk. If one functioning allele and one deficient allele are identified, the patient's phenotype with respect to UGT1A1 may be identified as increased risk. If two deficient alleles are identified, the patient's phenotype with respect to UGT1A1 may be identified as high risk. In some embodiments, the patient's phenotype with respect to each event/response gene may be identified.

With continued reference to FIG. 3, the process 300 may additionally include drug evaluation 330. Drug evaluation 330 may evaluate one or more drugs with respect to the patient genotype and phenotype data identified in the patient genotype evaluation 320. Drug evaluation 330 may include, for each of one or more drugs, determining a metabolizer score, classifying the drug, considering event/response genes, adjusting the drug classification, and determining one or more clinical annotations.

Drug evaluation 330 may include determining or calculating a drug score for one or more drugs. The drug score may be determined or calculated based on the phenotypes determined for the one or more metabolizer genes. To determine a score for a particular drug, a weight may be assigned to each of the one or more metabolizer genes with respect to the gene's effectiveness in metabolizing the particular drug. For example, where a first metabolizer gene is known or expected to be a primary contributor to the metabolization of a particular drug, and a second metabolizer gene is known or expected to be a minor contributor to the metabolization of such drug, the first metabolizer gene may be assigned a weight of approximately 75%, and the second metabolizer gene may be assigned a weight of approximately 25%. In other embodiments, the metabolizer genes may be assigned any other suitable weight. Moreover, where more than two metabolizer genes contribute to the metabolization of the particular drug, the multiple genes may be assigned other suitable weights. For example, where three metabolizer genes are involved in the metabolization of a particular drug, and where two of the three genes are known or expected to be primary contributors to metabolization of the drug, the two primary contributors together may be assigned a weight of approximately 75%, which may be divided between the two genes, thus assigning a weight of 37.5% to each of the two genes. Generally, the sum of the assigned weights for each of the genes that contribute to the metabolization of a particular drug may be approximately 100%. An example of metabolizer gene weight calculations is described below:

For drug A, where curated knowledge suggests that CYP2D6 and CYP3A4 are major metabolizers, and that CYP3A5 contributes only in a minor way to the metabolization, the weights assigned to each metabolizer gene may be, according to some embodiments:

CYP2D6+CYP3A4=75%
75%/2=37.5%
CYP2D6=CYP3A4=37.5%

CYP3A5=25%

Metabolization weights for a particular drug may be adjusted based on other curated knowledge factors as well. For example, curated knowledge may suggest that where CYP3A4 is identified as a metabolizer gene for any drug, CYP3A5 may be implicated as well. Thus, for example, where CYP3A4 is assigned a weight, the weight may be divided between CYP3A4 and CYP3A5. Other adjustments to metabolizer weights may be made based on curated knowledge as well.

Based on the one or more assigned or calculated metabolization weights, a drug score may be determined. The drug score may be a numerical score in some embodiments. In some embodiments, the drug score may be calculated by applying the gene weights for each identified metabolizer gene for a particular drug to the phenotype for each gene. For example, where a metabolizer gene is identified as contributing to the metabolization of a particular drug, the weight assigned to that gene may be multiplied by the gene's metabolizer score to achieve a weighted metabolizer score. The weighted metabolizer scores for each gene identified as contributing to the metabolization of a particular drug may be summed to find a drug score for the particular drug. An example calculation of a drug score is provided below.

CYP2D6
Assigned weight: 37.5%
Metabolizer score: 2.4
Weighted metabolizer score: 37.5%×2.4=0.9

CYP3A4
Assigned weight: 37.5%
Metabolizer score: 1.5
Weighted metabolizer score: 37.5%×1.5=0.56

CYP3A5
Assigned weight: 25%
Metabolizer score: 3.2
Weighted metabolizer score: 25%×3.2=0.8

Drug score=0.9+0.56+0.8=2.26

The drug score for a particular drug may generally indicate that drug's compatibility with the patient's genotype. In some embodiments, a relatively moderate drug score may indicate that a drug may be suitable for, or compatible with, the patient and thus may be used by the patient as directed. Moreover, in some embodiments, a relatively high or relatively low drug score may indicate that a drug may not be suitable for, or compatible with, the patient and thus should be used by the patient with great caution.

Drug evaluation 330 may additionally include classifying one or more drugs. That is, the one or more drugs may be classified according to its compatibility with, or suitability for, the patient. The one or more drugs may be classified based on, for example, a comparison to the patient's one or more metabolizer genes and/or a comparison to the patient's one or more event/response genes. For example, in some embodiments, the one or more drugs may be assigned a metabolizer classification based on comparison with the one or more metabolizer genes. Additionally or alternatively, the one or more drugs may be assigned an event/response classification based on a comparison with the one or more event/response genes. Moreover, in some embodiments, the one or more drugs may be assigned an overall drug classification. The overall drug classification may be a combination of the metabolizer classification and event/response classification in some embodiments. In other embodiments, the overall drug classification may be one of the metabolizer classification and event/response classification. In still other embodiments, an overall drug classification for the one or more drugs may be determined or calculated by other means.

The metabolizer classification, event/response classification, overall drug classification, and/or any other classification assigned to one or more drugs may be a color-coded classification in some embodiments. For example, a classification of “red” may indicate that a drug should be used with great caution. Similarly, a classification of “yellow” may indicate that a drug should be used with caution. Further, a classification of “green” may indicate that a drug may be used as directed. In other embodiments, the one or more classifications may be signified by a number, letter, symbol, title, or any other suitable identifier.

In some embodiments, a drug may be assigned a metabolizer classification based on its drug score. In some embodiments, a relatively moderate drug score may result in a “green” classification assigned to the drug, wherein the drug may be used as directed. In some embodiments, a relatively low or relatively high drug score may indicate that a drug may be classified as a “red” drug, wherein the drug may be used with great caution. Drugs having drug scores between the red and green zones may be classified as “yellow” drugs in some embodiments, wherein the drugs may be used with caution. In other embodiments, other drug scores may indicate other classifications. For example, a drug score of between approximately 0 and approximately 0.75 or a drug score of more than approximately 3 may result in a drug classification of “red;” a drug score of between approximately 0.75 and approximately 1.75 or between approximately 2.25 and approximately 3 may result in a drug classification of “yellow;” and a drug score of between approximately 1.75 and approximately 2.25 may result in a drug classification of “green.” In other embodiments, other suitable drug score ranges may indicate red, yellow, or green classifications or other classifications. In some embodiments, the drug classification based on one or more metabolizer genes may be a metabolizer classification or first classification.

In addition to or alternative to the metabolizer classification, one or more drugs may be assigned an event/response classification based on comparison of the drug to one or more event/response genes of the patient. As described above, some event/response genes may indicate a possibility or risk for various adverse effects, increased or decreased effectiveness, and/or other reactions to one or more drugs. For example, where an event/response gene for the patient indicates particular toxicity or another adverse effect with respect to a particular drug, that drug may be categorized as red, wherein the drug may be used with great caution by the patient. For example, as described above, two deficient alleles for the gene UGT1A1 may indicate a high risk of hyperbilirubinemia, toxicity, and/or other adverse effects with respect to particular drugs. Each of these particular drugs may be classified as red for the patient having two deficient UGT1A 1 alleles. Where the patient's UGT1A1 alleles indicate no or minimal risk for toxicity for the particular drugs, the particular drugs may be classified as green or yellow. In some embodiments, each event/response gene may be evaluated with respect to one or more drugs. In some embodiments, a drug may receive multiple event/response classifications based on its having an interaction with multiple event/response genes.

In some embodiments, a drug may be assigned an overall drug classification. The overall drug classification for a drug may be based, at least in part, on the metabolizer classification for the drug and the event/response classification(s) for the drug. For example, in some embodiments, the metabolizer classification and event/response classification(s) for a drug may be compared to determine a highest or most cautious classification assigned to the drug. In some embodiments, the highest or most cautious classification assigned to the drug may govern as the overall classification for the drug. For example, where a metabolizer classification assigned to the drug is green, but an event/response classification assigned to the drug is red, the overall drug classification for the drug may be designated as red. In other embodiments, the metabolizer classification and event/response classification(s) assigned to a drug may be averaged or combined. For example, where a metabolizer classification assigned to the drug is green, and an event/response classification assigned to the drug is red, the overall classification for the drug may be designated as yellow. In other embodiments, an overall drug classification may be calculated or determined by other means.

In some embodiments, drug evaluation 330 may include determining one or more clinical annotations. Clinical annotations may include data related to a particular drug that may be relevant for patient use of the drug. For example, clinical annotations may include dosing guidelines provided by the FDA or another source. The dosing guidelines may be generally applicable to the drug and/or may be particular to the patient's particular genotype. In some embodiments, clinical annotations may include dosing guidelines from multiple sources. Clinical annotations may additionally or alternatively include data related to genomic biomarkers. For example, in some cases, the FDA publishes information on genomic biomarkers limiting drug indications to patients that exhibit specific biomarkers. In such cases, clinical annotations may include an indication that additional testing may be required before administration of a particular drug. Other clinical annotations may include side effect warnings, effectiveness warnings, which may be generally related to use of a drug or may be specific to the patient's genotype. For example, a clinical annotation may include a warning of increased risk of toxicity or side effects where a patient's phenotype indicates a potential for increased conversion of a drug to its active metabolite(s).

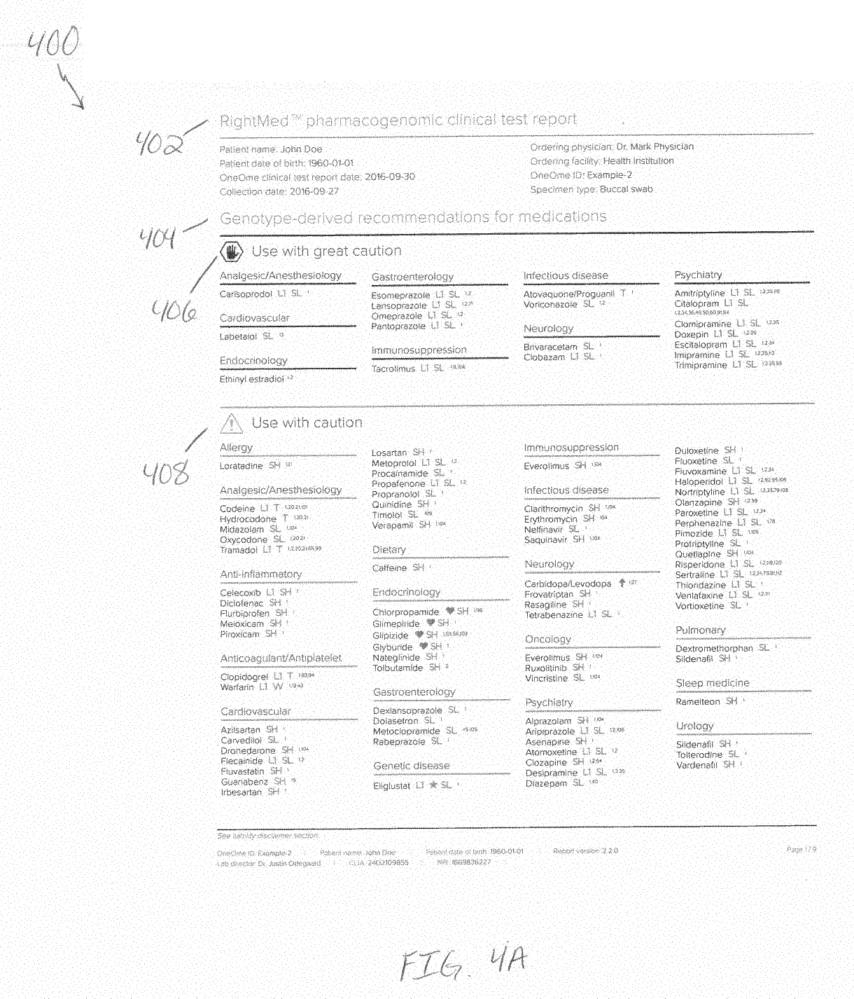

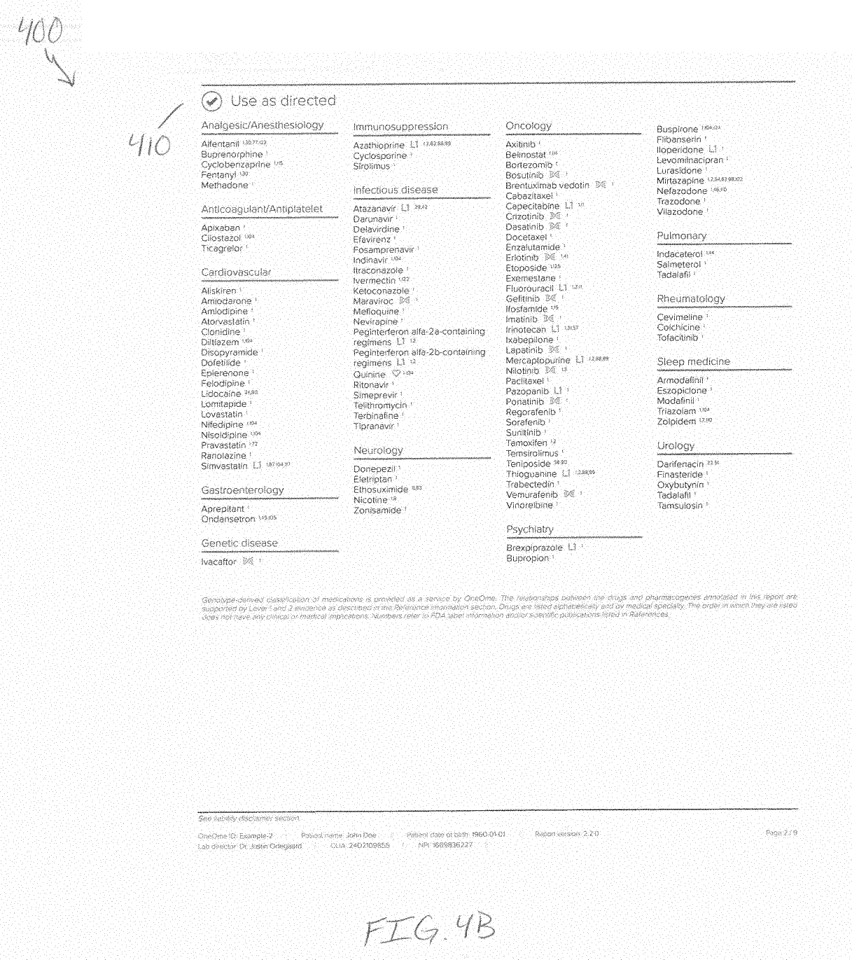

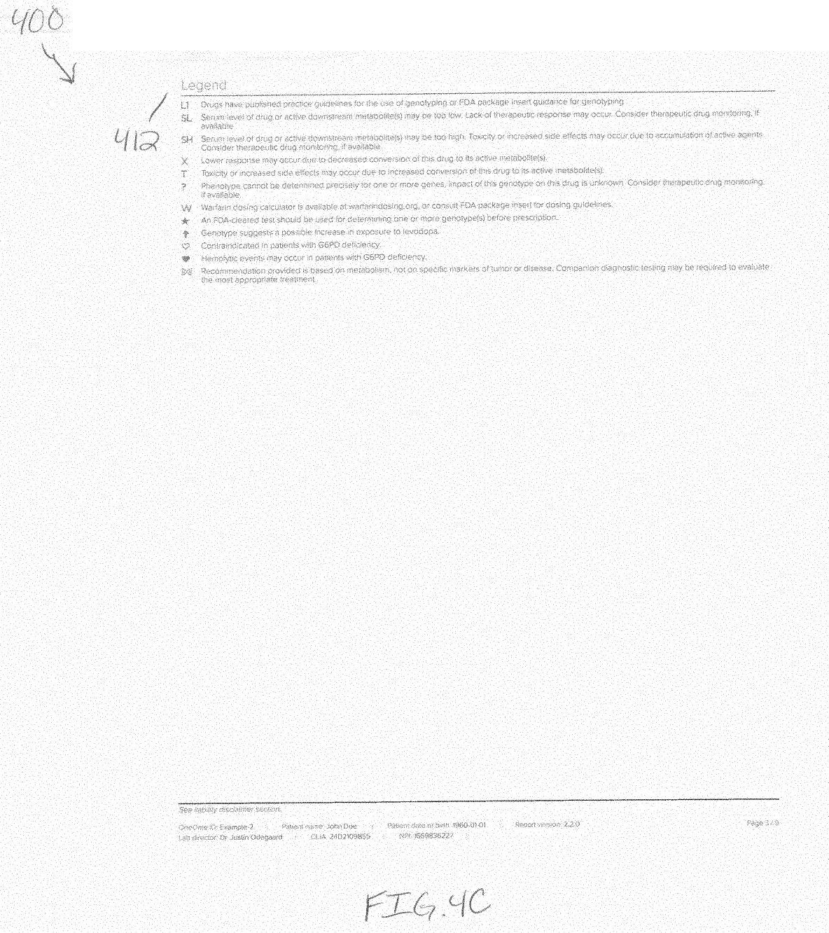

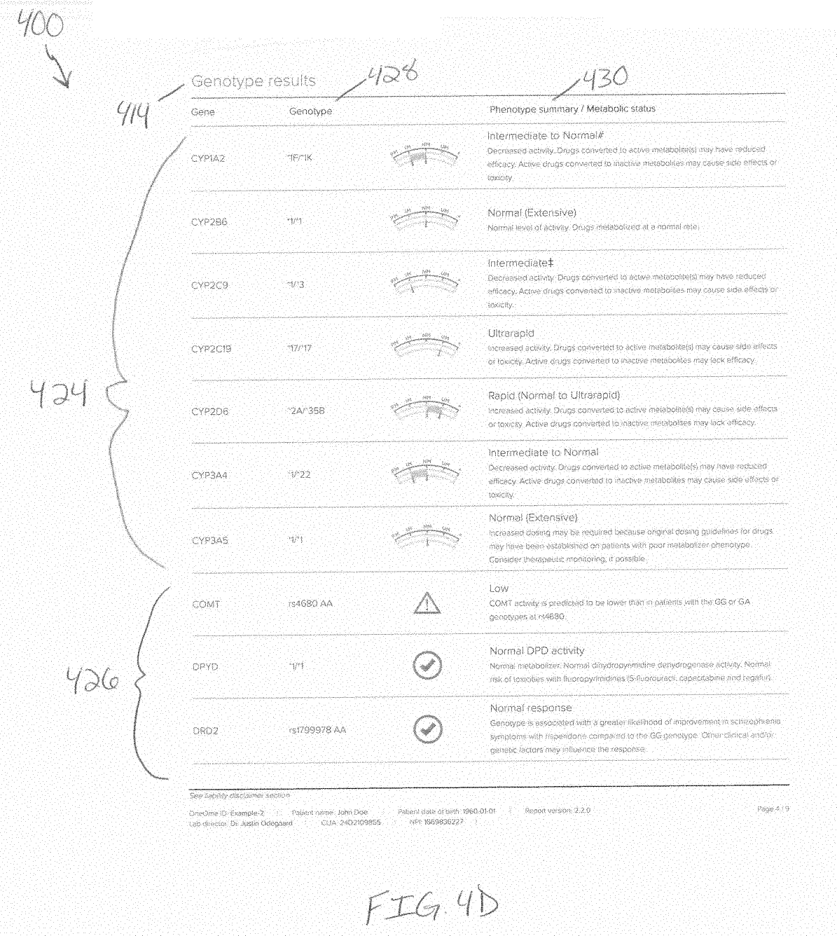

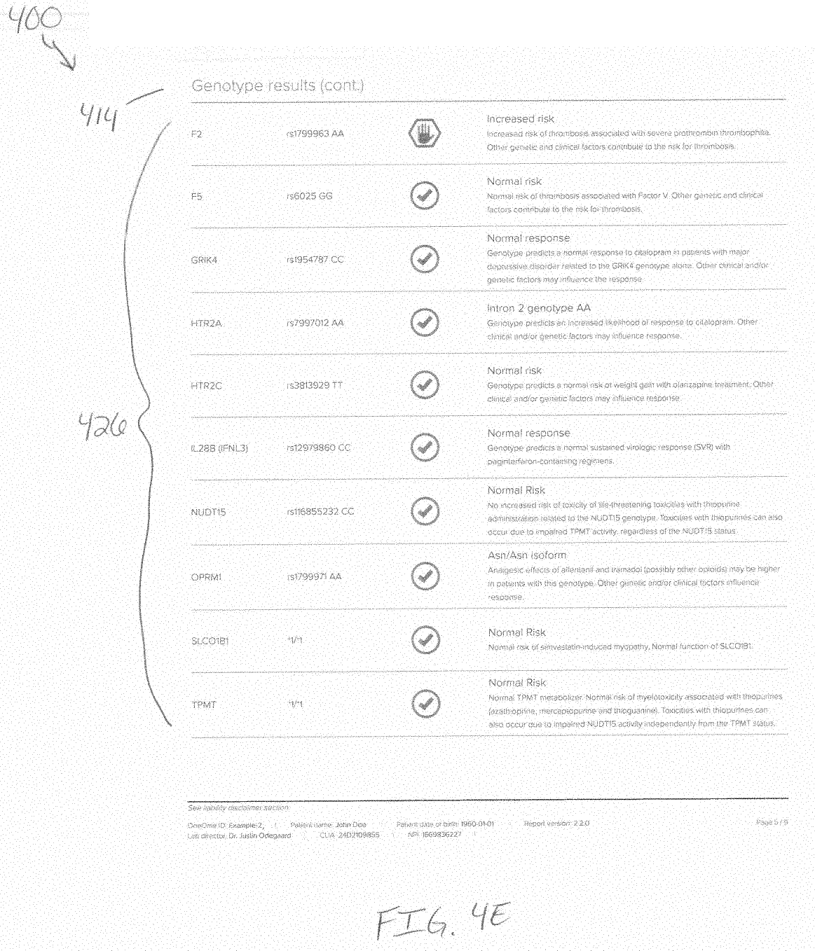

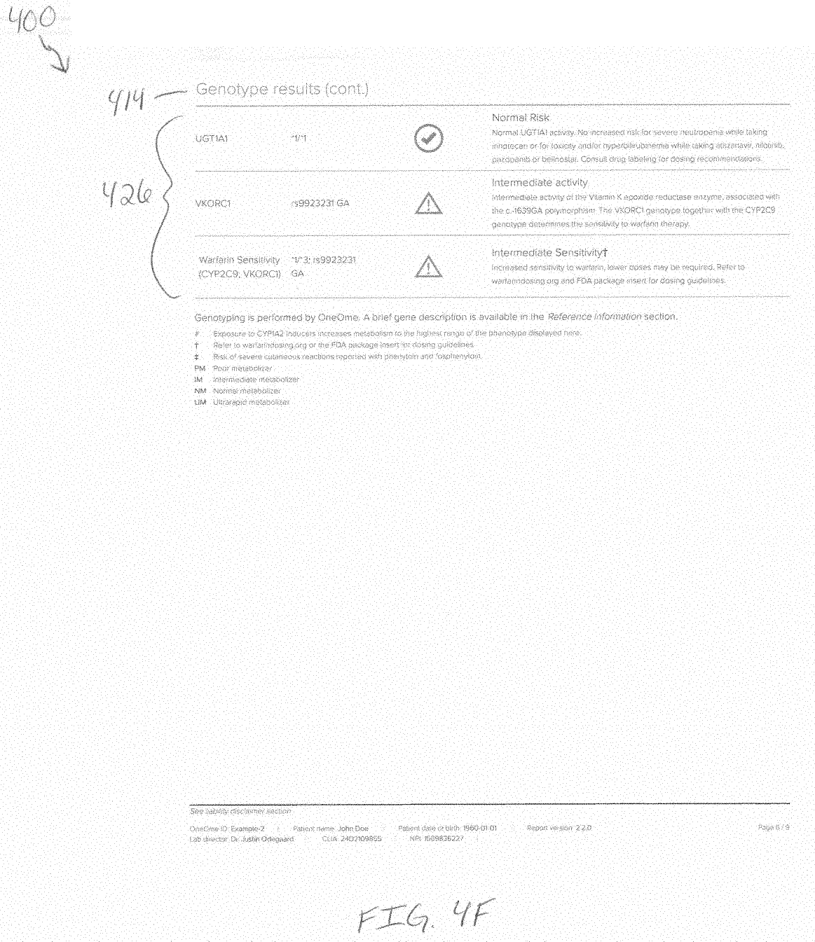

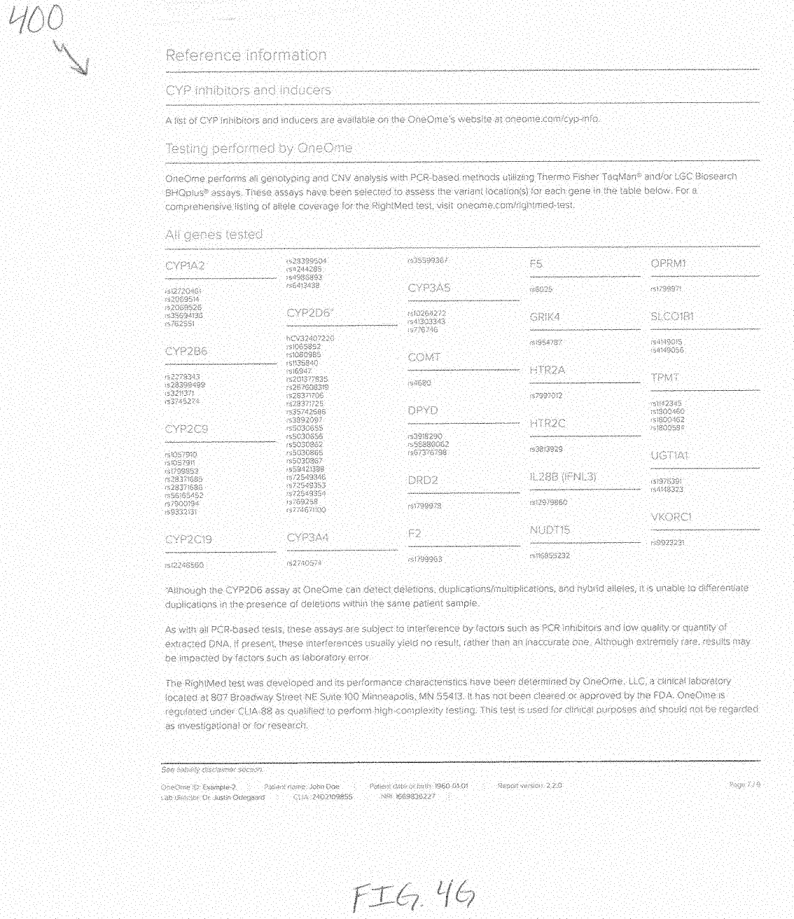

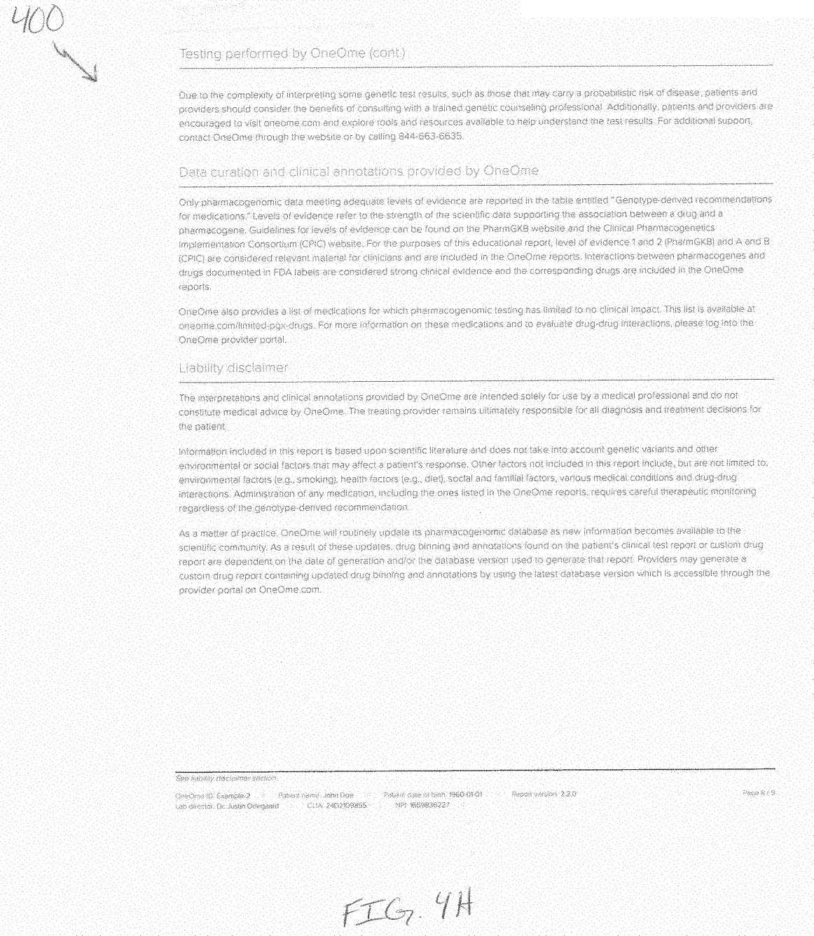

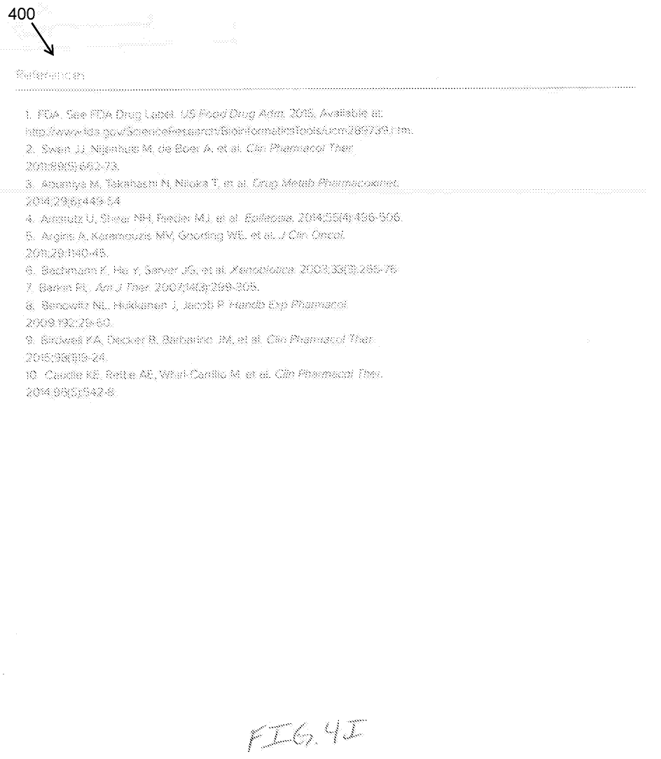

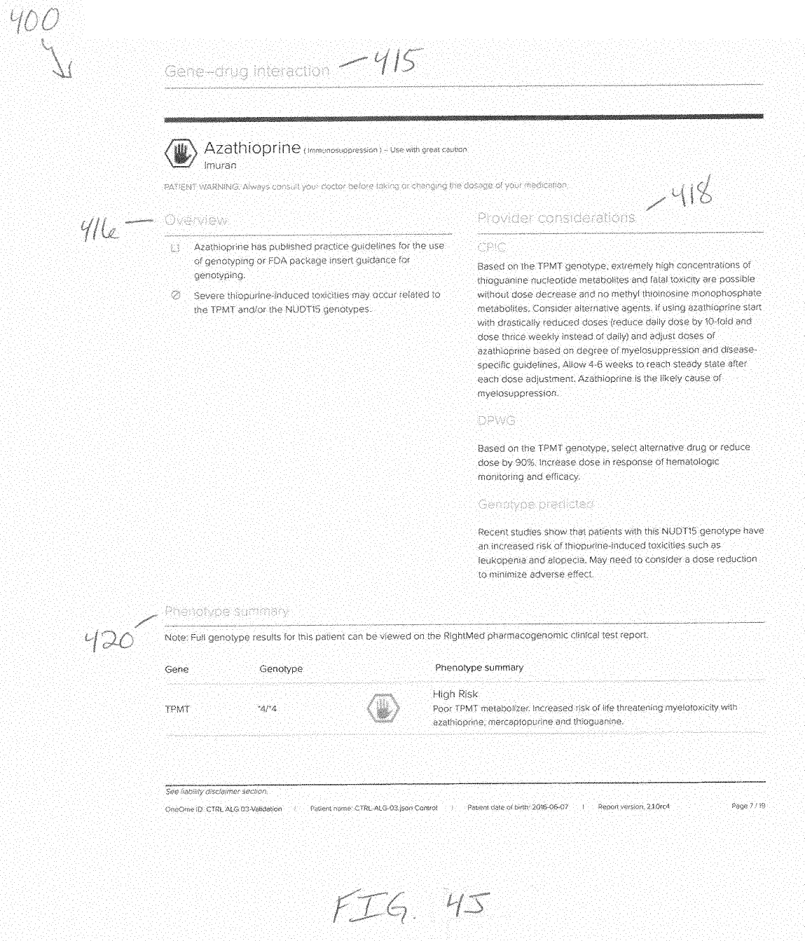

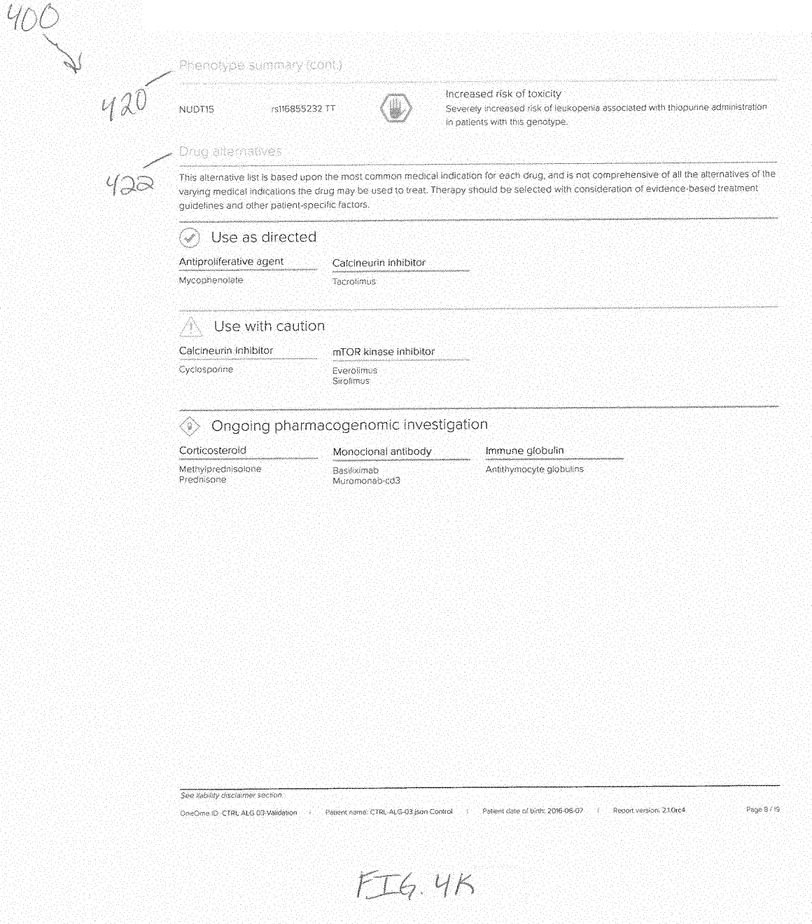

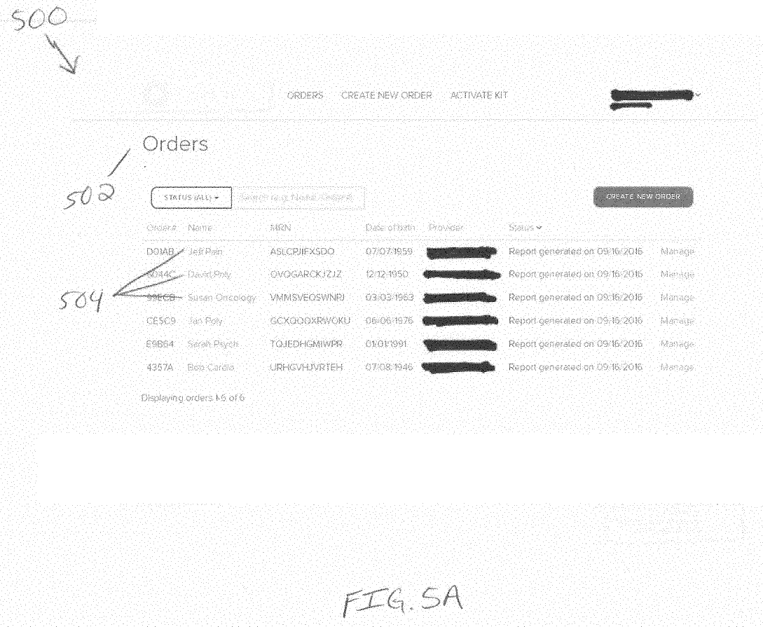

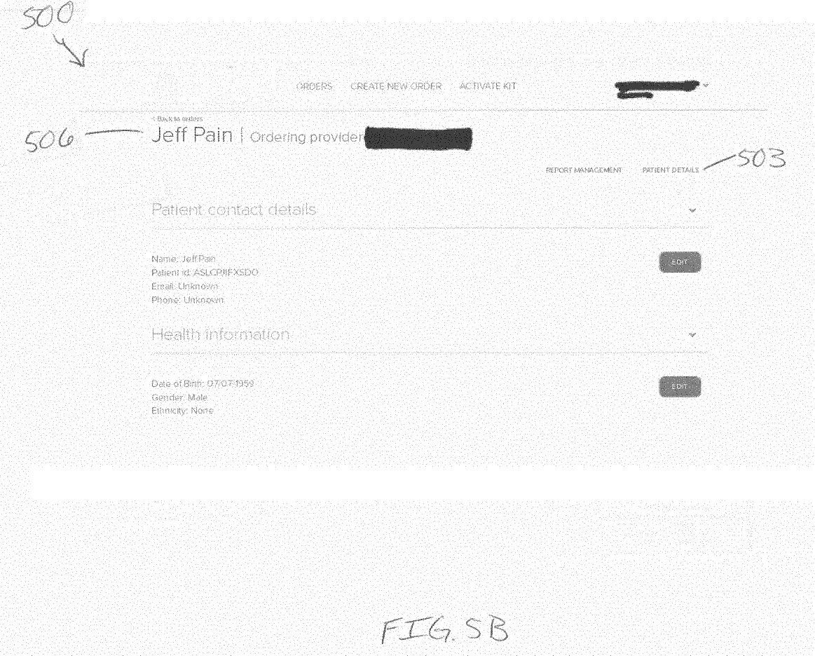

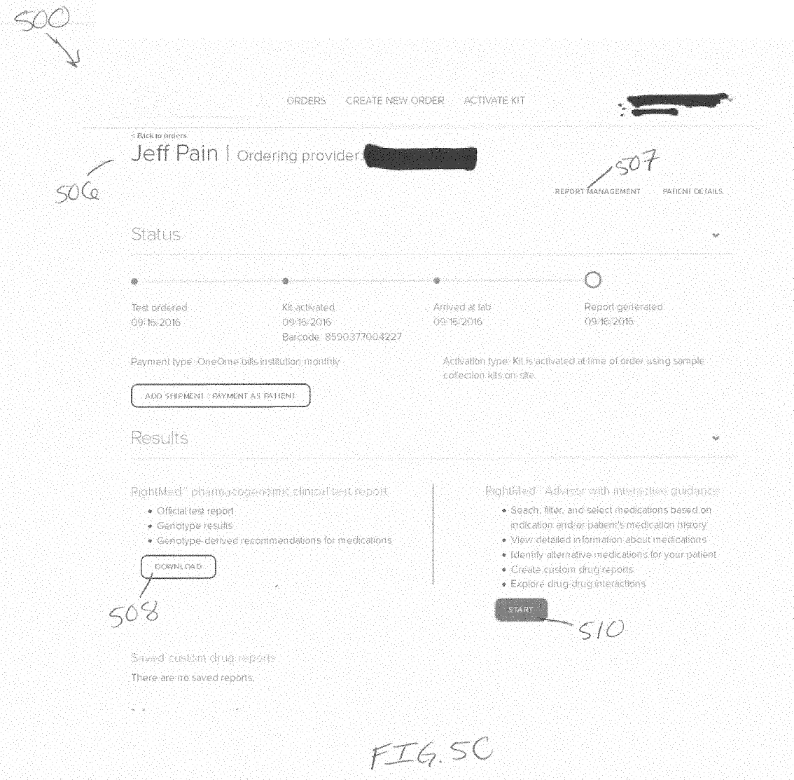

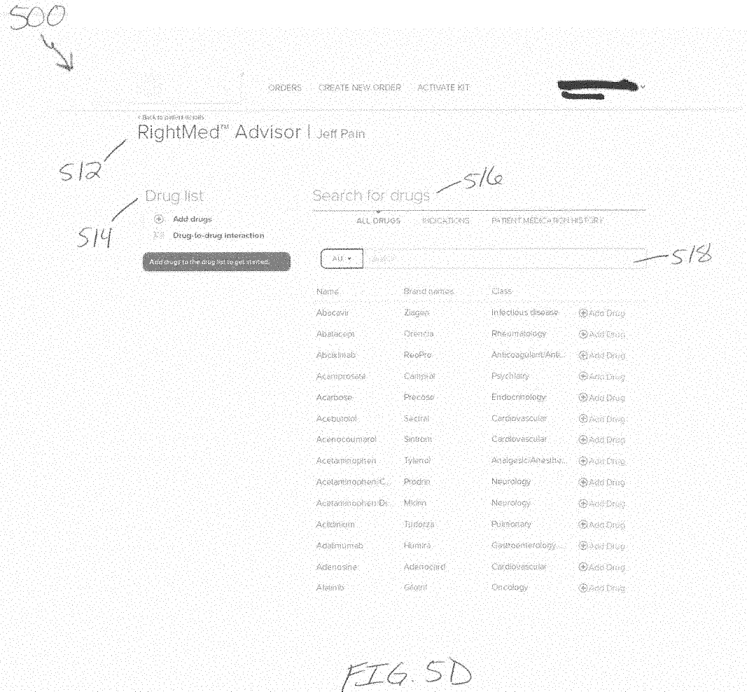

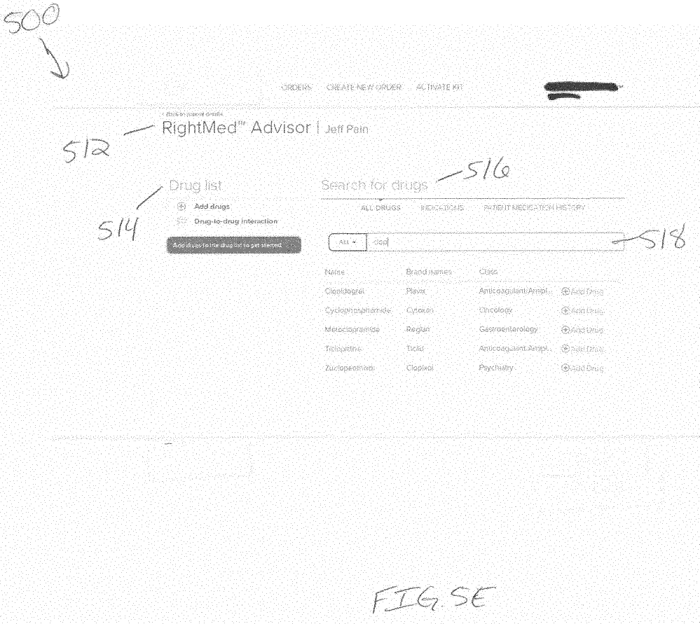

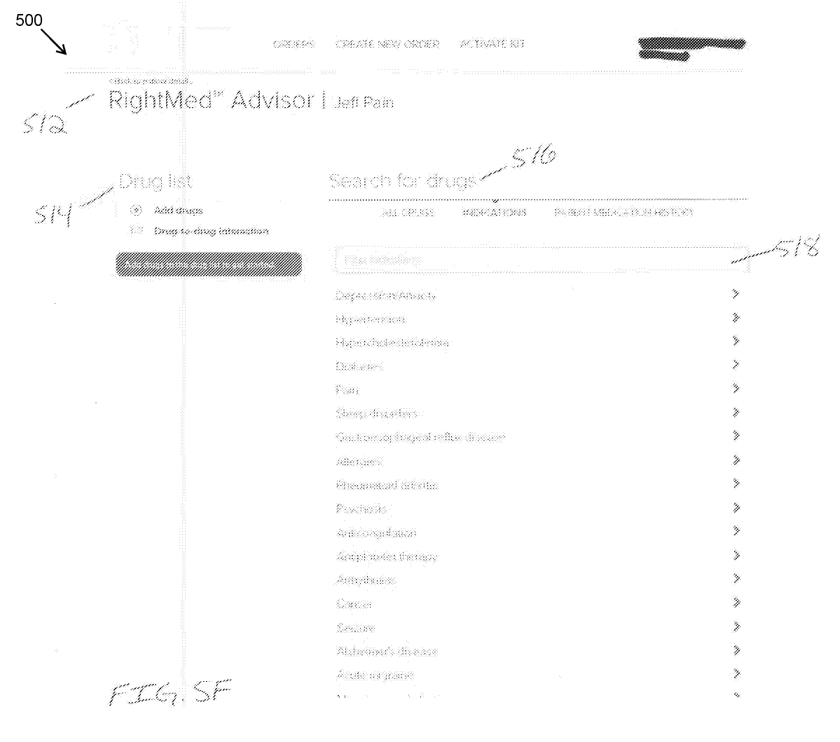

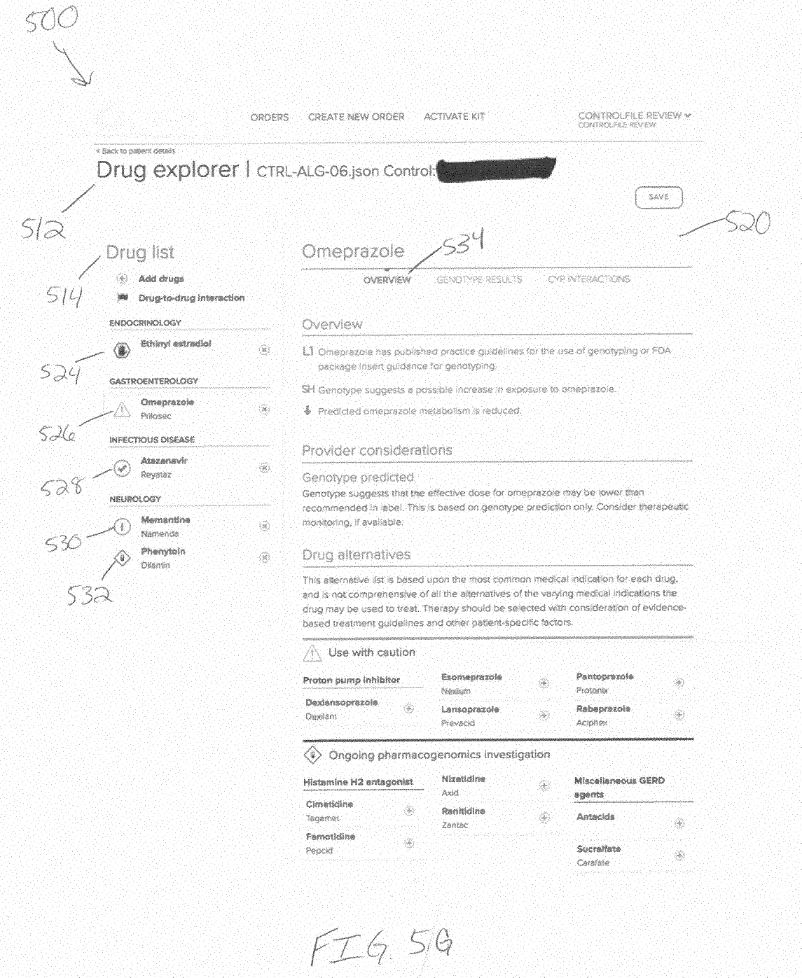

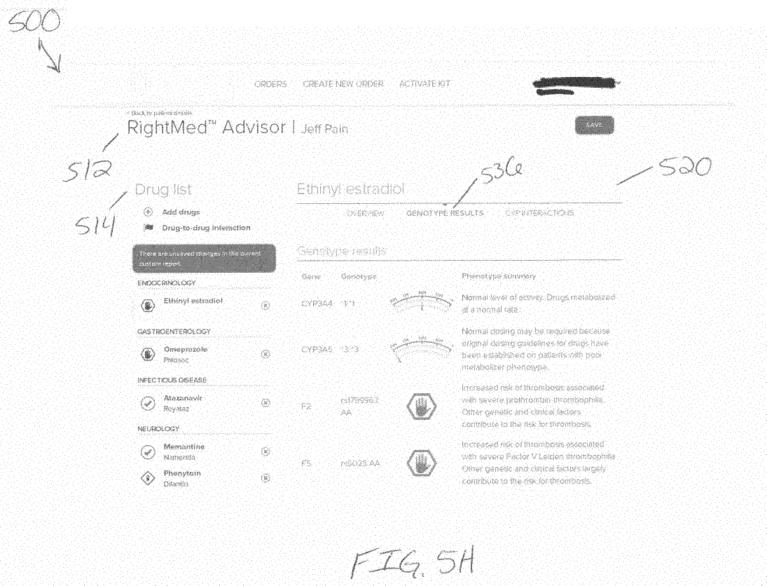

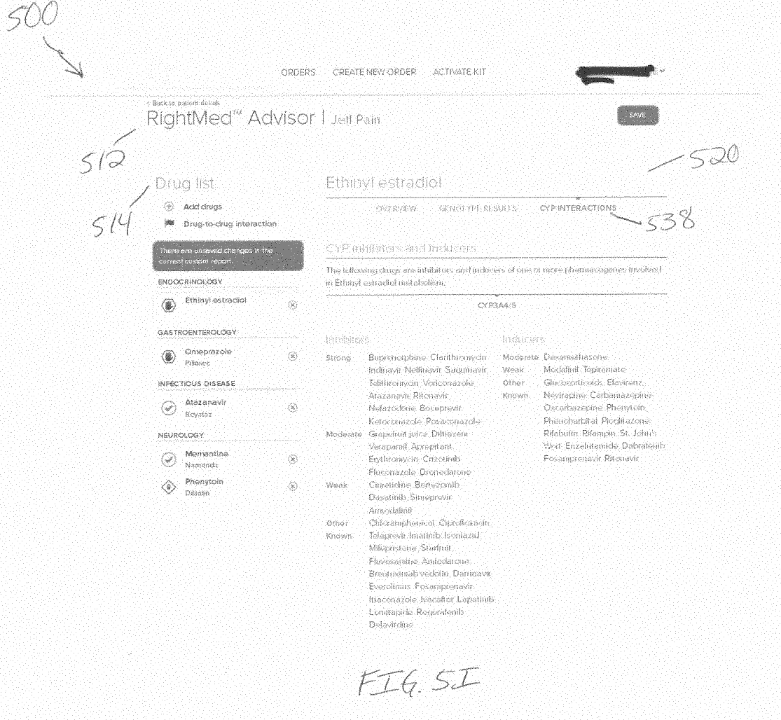

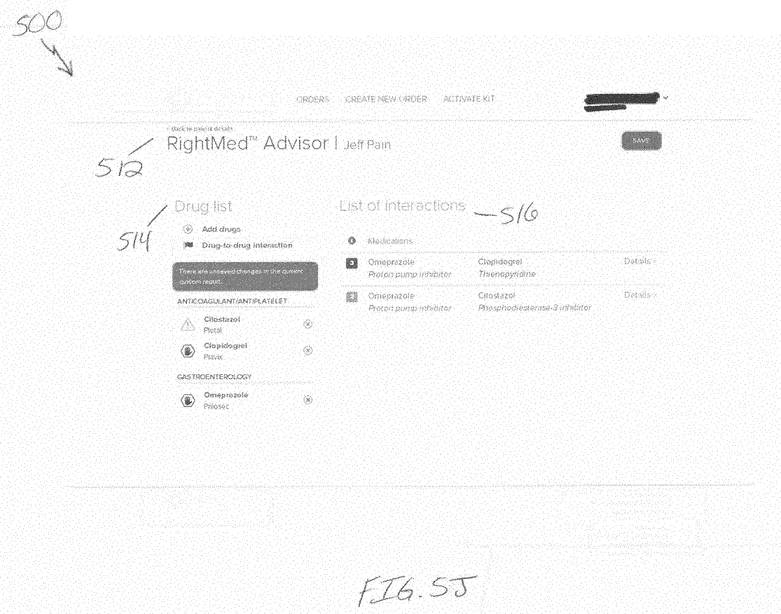

With continued reference to FIG. 3, the process 300 may additionally include providing one or more recommendations 340, wherein one or more recommendations related to one or more drugs may be provided to a doctor, other health care professional, or to the patient. The recommendations may include providing a report detailing the recommendation(s). The report may be provided in any suitable form, including digitally or physically. FIGS. 4A-4K, discussed below, illustrate an example of a recommendations report, according to at least one embodiment. Moreover, in some embodiments, one or more recommendations may be provided using an interactive terminal. FIGS. 5A-5J, discussed below, illustrate an example of an interactive reporting terminal, according to at least one embodiment.

FIGS. 4A-4K illustrate a recommendations report 400 according to at least one embodiment. The recommendations report 400 may generally provide one or more recommendations regarding a patient's compatibility with one or more drugs. As shown in FIG. 4A, the recommendations report 400 may include a report data section 402 and a medications recommendation section 404. The report data section 402 may provide one or more patient details such as name, identifier, date of birth, or details; and/or report details such as date of report, report requesting entity, report generating entity, and/or other details. The medication recommendations section 404 may provide recommendations for the patient with respect to the one or more drugs that were compared with the patients genotype(s) and/or phenotype(s). For example, in some embodiments, the medication recommendations section 404 may provide a list of one or more medications sorted by their assigned overall classification, as described above. That is, one or more drugs may be listed under a classification of “use with great caution” 406, one or more drugs may be listed under a classification of “use with caution” 408, and as shown in FIG. 4B, one or more drugs may be listed under a classification of “use as directed” 410. In other embodiments, the one or more drugs may be listed according to their assigned metabolizer classification and/or assigned event/response classification, as described above. The medication recommendation section 404 may generally allow a patient or health care provider to select an appropriate medication for treating the patient, while avoiding medications that may be incompatible with the patient's DNA. In some embodiments, the report 400 may provide additional information or guidance regarding at least some of the listed drugs. For example, the report 400 may provide an indication regarding low or high serum levels, the availability of published dosing guidelines, active metabolites, potential risk for side effects or contra-indication warnings, or where additional testing is advised or suggested. Moreover, the report 400 may provide an indication regarding incomplete genotype and/or phenotype identification. For example, where an allele pair could not be determined or was estimated, the report 400 may provide an indication of this information. In some embodiments, such indicators or guidance may be provided by a plurality of symbols arranged next to one or more applicable drugs. A legend 412, as shown on FIG. 4C may provide a key to each symbol.

In some embodiments, as shown in FIG. 4D, the report 400 may have a genotype results section 414. The genotype results section 414 may provide a list of one or more genes evaluated with respect to the patient's DNA. For example, the genotype results section 414 may provide a list of one or more metabolizer genes 424 and/or one or more event/response genes 426 evaluated with respect to the patient's DNA. For each metabolizer gene 424 and/or event/response gene 426, the report 400 may list an identified genotype 428 and/or an identified phenotype 430. In some embodiments, the genotype 428 for each gene listed in the genotype results section 414 may be identified by a star allele or other standard nomenclature. As described above, the identified phenotype 430 for a metabolizer gene 424 may be the expected metabolic status with respect to metabolizing a drug. As further described above, the identified phenotype 430 for an event/response gene 424 may be the expected level of risk of side effects, effectiveness, and/or other physiologic responses the patient may experience in response to one or more drugs. As additionally shown in FIGS. 4D, 4E, and 4F, in some embodiments, the genotype results section 414 may provide a graphic or iconic indicator of each gene's phenotype 430. For example, for the one or more metabolizer genes 424, the report 400 may provide a gauge icon indicating how rapidly the gene is expected to metabolize a drug. Additionally or alternatively, for the one or more event/response genes 426, the report 400 may provide an icon indicating a level of caution that should be taken based on the gene's phenotype.

As shown in FIGS. 4G, 4H, and 4I, other information or guidance may be provided in the report 400 as well. For example, as shown in FIG. 4G, the report 400 may provide a list of one or more CYP inhibitors and/or CYP inducers. CYP inhibitors and inducers may include one or more drugs, foods, or other substances that may affect the ability of the metabolizer genes to metabolize one or more drugs. Moreover, the report 400 may provide a list of all genes identified in the patient's DNA, including in some embodiments a list of SNP locations identified. The report 400 may additionally include information related to the process of genotype identification, phenotype identification, and/or drug evaluation. As shown in FIG. 4I, in some embodiments, the report 400 may provide a list of references from which knowledge was curated to determine the recommendations, guidance, and other information contained within the report.

In some embodiments, the report 400 may data specific to one or more individual drugs. As shown for example in FIGS. 4J and 4K, in some embodiments, the report 400 may include a gene-drug interaction section 415 providing detailed recommendations and other data for a particular list of drugs. The gene-drug interaction section 415 may be provided where requested by the patient or a healthcare professional in some embodiments. For example, a patient or healthcare professional may request a drug-patient interaction section 415 for one or more drugs the patient may be currently taking, may consider taking in the future, or which the healthcare professional may consider prescribing for the patient. The drug-gene interaction section 415 may include an overview section 416, a provider considerations section 418, a phenotype summary 420, and a drug alternatives section 422 in some embodiments. The overview section 416 may provide general information about the particular drug including, for example, general warnings or risks. The provider considerations section 418 may provide one or more guidelines or other information related to prescribing the particular drug. The phenotype summary 420 may provide a list of one or more genes, such as event/response genes, that may affect the patient's response to the particular drug. In some embodiments, one or more metabolizer genes may be listed as well. For each listed event/response and/or metabolizer gene, the report 400 may provide an identified genotype and/or phenotype. Moreover, in some embodiments, an icon or graphic indicating a level of risk or caution may be provided for each listed gene. As shown in FIG. 4K, the drug alternatives section 422 may provide a list of one or more similar, bioequivalent, or related drugs that a patient or healthcare professional may consider instead of the particular drug. The alternative drugs may be categorized as “use as directed,” “use with caution,” “use with great caution,” and/or “ongoing pharmacogenomics investigation” in some embodiments.

FIGS. 5A-5J illustrate an interactive reporting terminal 500 according to at least one embodiment. The reporting terminal 500 may generally provide interactive and on-demand access for one or more users to data otherwise available in a report 400. That is, once a patient's DNA is evaluated for genotype(s), phenotype(s), and compatibility with one or more drugs, the patient's information and one or more recommendations may be available via the reporting terminal 500. In some embodiments, the reporting terminal 500 may be configured to be accessible by a doctor, nurse, laboratory technician, and/or other healthcare professional user. In other embodiments, the terminal 500, or at least portions thereof, may additionally or alternatively be accessible by a patient user.

As shown in FIG. 5A, where the terminal 500 is configured for access by a healthcare professional, for example, the terminal may have an orders page 502 providing a list of patients 504 for which one or more drug recommendations has been made or requested. The orders page 502 may illustrate whether a patient's DNA has been received, is currently under consideration, and/or whether a report or one or more recommendations has been generated. From the orders page 502, a user may access detailed information for a patient 504. By selecting a patient 504, a user may access a patient information page 506, as shown in FIGS. 5B and 5C. As shown in FIG. 5B, the patient information page 506 may include a patient details tab 503, wherein contact information, health information, and/or other information or data about the patient may be listed or maintained. Moreover, as shown in FIG. 5C, the patient information page 506 may include a report management tab 507 where a user may view the status of one or more reports regarding the patient. Where a report, or one or more medication recommendations, has been generated, the patient information page 506 may provide options to interactively view the report or recommendations 510, and/or to download a copy of the report or recommendations 508.

FIG. 5D illustrates an example of an interactive report page 512. The interactive report page 512 may provide detailed medication recommendations and/or other information that may otherwise be available in a report 400. The interactive report page 512 may provide the information in an easily navigable and viewable format. For example, the interactive report page 512 may have a drug list panel 514. The drug list panel 514 may generally remain visible as the user navigates the interactive report in some embodiments. In some embodiments, a user may have the ability to add one or more drugs to the drug list panel 514 so as to view information related to the patient's compatibility with each drug. In some embodiments, the interactive report page 512 may have a search option 516 wherein a user may search for one or more drugs to add to the drug list panel 514. The search option 516 may include a search field 518. As shown in FIG. 5D, a user may view a list of drug names or search for drugs by name in some embodiments. The user may scroll through the available drugs or use the search field 518. In some embodiments, the search field 518 may be configured to auto-complete an indication or drug name or identifier based on a first few characters typed by the user, as shown for example in FIG. 5E. As shown in FIG. 5F, a user may additionally or alternatively have the option to view or search for drugs by indication. The user may scroll through the various indications or may use the search field 518 to search for a particular indication, such as depression, hypertension, diabetes, pain, and/or other indications for which a drug may be used. Additionally or alternatively, the user may have the option to search for drugs within the patient's medication history, which may leverage integration to a third party service provider such as SureScripts, for example. In this way, the user may scroll through the patient's medication history or may use the search field 518 to search for a particular drug name or other identifier within the patient's medical history.

A user may add one or more drugs from the search option 516 to the drug list panel 514, as shown in FIG. 5G. In some embodiments, drugs added to the drug list panel 514 may be displayed with a graphic, iconic, and/or color indicating a level of caution that should be taken based on the patient's phenotype and/or genotype. For example, as shown in FIG. 5G, a first icon 524, which may be a red icon in some embodiments, may indicate that a patient should use great caution when using the particular medication. A second icon 526, which may be a yellow icon in some embodiments, may indicate that a patient should use caution when using the particular medication. A third icon 528, which may be a green icon in some embodiments, may indicate that a patient may take the particular medication as directed. Moreover, in some embodiments, a fourth icon 530, which may be a blue icon in some embodiments, may indicate limited pharmacogenomic impact with respect to the particular medication. A fifth icon 532, which may be a grey icon in some embodiments, may indicate that one or more investigations or studies may be ongoing and/or that additional investigation and/or information may be needed with respect to the particular drug. In other embodiments, other icons, colors, or other indicators may identify additional or alternative levels of caution or other properties with respect to particular drugs.

A user may view detailed report information about a drug added to the drug list panel 514, at least some of which may be specific to the patient's genotype(s) and/or phenotype(s). FIG. 5G shows a drug detail view 520. The drug detail view 520 may provide access to data described above with respect to the drug-gene interaction section 415 of a report 400. For example, the drug detail view 520 may have a drug overview tab 534 providing general information about the drug and the patient's use of the drug. The overview tab 534 may include an overview section with information such as general warnings or risks; a provider considerations section providing one or more guidelines or other information related to prescribing the particular drug; and a drug alternatives section providing a list of one or more similar, bioequivalent, or related drugs that a patient or healthcare professional may consider instead of the particular drug. The alternative drugs may be categorized as “use as directed,” “use with caution,” “use with great caution,” “pharmacogenomics of limited impact,” and/or “ongoing pharmacogenomics investigation” in some embodiments. As shown in FIG. 5H, the drug detail view 520 may additionally include a genotype results tab 536 providing a list of one or more genes, such as event/response genes that may affect the patient's response to the particular drug, and/or metabolizer genes that may be involved in metabolization of the drug, along with an identified genotype and/or phenotype for each gene. Moreover, in some embodiments, an icon or graphic indicating a metabolizer type or level of risk or caution may be provided for each listed gene. As additionally shown in FIG. 5I, the drug detail view 520 may include a CYP interactions tab 538 providing a list of CYP inhibitors and inducers for the particular drug.

In some embodiments, the interactive report page 512 may provide a list of drug-drug interactions. For example, after a user adds two or more drugs to the drug list panel 514, the user may have the ability to view whether there are any interactions between the two or more drugs. FIG. 5J illustrates a list of drug-drug interactions 516 accessible via the interactive report page 512.

In some embodiments, the terminal 500 may provide a user with the option to create a customized drug report for a patient. That is, for example, after adding one or more drugs to the drug list panel 514, a user may have the option to create a custom report including recommendations for the patient particular to those selected drugs in the drug list panel. In addition to or alternative to the information described above with respect to a report 400, a customized report may include a drug-gene interaction section for each drug selected by the user for inclusion in the customized report and/or information regarding interactions among the selected drugs.

In the present disclosure, systems, methods, and processes for providing one or more patient-specific drug recommendations have been described. The one or more recommendations may be made based on a comparison of the patient's DNA, or DNA data, including one or more genotypes and/or phenotypes of the patient, to one or more drugs or other substances. The one or more recommendations may further be made based on one or more known or expected drug-gene relationships. Drug-gene relationships may be documented in a curated knowledge database. The curated knowledge database may include data derived from a variety of sources. In some embodiments, the data derived from a variety of sources may be subject to a scale of evidence criteria to determine whether it should be used to provide medication recommendations. In some embodiments, the one or more patient-specific drug recommendations may be updated. For example, as new data is added to the curated knowledge database, the patient's genotype(s) and/or phenotype(s) may be re-compared to one or more drugs and/or may be compared to one or more additional drugs. In this way, the patient-specific recommendation(s) may be updated or revised. In some embodiments, the patient's DNA data may be compared to the one or more drugs continuously, intermittently, at intervals, or based on a request. For example, a patient or healthcare professional may request that a patient's one or more recommendations be updated or revised. In some embodiments, a patient's DNA data may be compared automatically when the curated knowledge database acquires new drug-gene relationship data. In other embodiments, the patient's DNA data may be compared manually or automatically at any suitable time.

For purposes of this disclosure, any system described herein may include any instrumentality or aggregate of instrumentalities operable to compute, calculate, determine, classify, process, transmit, receive, retrieve, originate, switch, store, display, communicate, manifest, detect, record, reproduce, handle, or utilize any form of information, intelligence, or data for business, scientific, control, or other purposes. For example, a system or any portion thereof may be a minicomputer, mainframe computer, personal computer (e.g., desktop or laptop), tablet computer, mobile device (e.g., personal digital assistant (PDA) or smart phone) or other hand-held computing device, server (e.g., blade server or rack server), a network storage device, or any other suitable device or combination of devices and may vary in size, shape, performance, functionality, and price. A system may include volatile memory (e.g., random access memory (RAM)), one or more processing resources such as a central processing unit (CPU) or hardware or software control logic, ROM, and/or other types of nonvolatile memory (e.g., EPROM, EEPROM, etc.). A basic input/output system (BIOS) can be stored in the non-volatile memory (e.g., ROM), and may include basic routines facilitating communication of data and signals between components within the system. The volatile memory may additionally include a high-speed RAM, such as static RAM for caching data.

Additional components of a system may include one or more disk drives or one or more mass storage devices, one or more network ports for communicating with external devices as well as various input and output (I/O) devices, such as a keyboard, a mouse, touchscreen and/or a video display. Mass storage devices may include, but are not limited to, a hard disk drive, floppy disk drive, CD-ROM drive, smart drive, flash drive, or other types of non-volatile data storage, a plurality of storage devices, a storage subsystem, or any combination of storage devices. A storage interface may be provided for interfacing with mass storage devices, for example, a storage subsystem. The storage interface may include any suitable interface technology, such as EIDE, ATA, SATA, and IEEE 1394. A system may include what is referred to as a user interface for interacting with the system, which may generally include a display, mouse or other cursor control device, keyboard, button, touchpad, touch screen, stylus, remote control (such as an infrared remote control), microphone, camera, video recorder, gesture systems (e.g., eye movement, head movement, etc.), speaker, LED, light, joystick, game pad, switch, buzzer, bell, and/or other user input/output device for communicating with one or more users or for entering information into the system. These and other devices for interacting with the system may be connected to the system through I/O device interface(s) via a system bus, but can be connected by other interfaces such as a parallel port, IEEE 1394 serial port, a game port, a USB port, an IR interface, etc. Output devices may include any type of device for presenting information to a user, including but not limited to, a computer monitor, flat-screen display, or other visual display, a printer, and/or speakers or any other device for providing information in audio form, such as a telephone, a plurality of output devices, or any combination of output devices.

A system may also include one or more buses operable to transmit communications between the various hardware components. A system bus may be any of several types of bus structure that can further interconnect, for example, to a memory bus (with or without a memory controller) and/or a peripheral bus (e.g., PCI, PCIe, AGP, LPC, etc.) using any of a variety of commercially available bus architectures.

One or more programs or applications, such as an interactive recommendations terminal, a web browser and/or other executable applications, may be stored in one or more of the system data storage devices. Generally, programs may include routines, methods, data structures, other software components, etc., that perform particular tasks or implement particular abstract data types. Programs or applications may be loaded in part or in whole into a main memory or processor during execution by the processor. One or more processors may execute applications or programs to run systems or methods of the present disclosure, or portions thereof, stored as executable programs or program code in the memory, or received from the Internet or other network. Any commercial or freeware web browser or other application capable of retrieving content from a network and displaying pages or screens may be used. In some embodiments, a customized application may be used to access, display, and update information. A user may interact with the system, programs, and data stored thereon or accessible thereto using any one or more of the input and output devices described above.

A system of the present disclosure can operate in a networked environment using logical connections via a wired and/or wireless communications subsystem to one or more networks and/or other computers. Other computers can include, but are not limited to, workstations, servers, routers, personal computers, microprocessor-based entertainment appliances, peer devices, or other common network nodes, and may generally include many or all of the elements described above. Logical connections may include wired and/or wireless connectivity to a local area network (LAN), a wide area network (WAN), hotspot, a global communications network, such as the Internet, and so on. The system may be operable to communicate with wired and/or wireless devices or other processing entities using, for example, radio technologies, such as the IEEE 802.xx family of standards, and includes at least Wi-Fi (wireless fidelity), WiMax, and Bluetooth wireless technologies. Communications can be made via a predefined structure as with a conventional network or via an ad hoc communication between at least two devices.

Hardware and software components of the present disclosure, as discussed herein, may be integral portions of a single computer or server or may be connected parts of a computer network. The hardware and software components may be located within a single location or, in other embodiments, portions of the hardware and software components may be divided among a plurality of locations and connected directly or through a global computer information network, such as the Internet. Accordingly, aspects of the various embodiments of the present disclosure can be practiced in distributed computing environments where certain tasks are performed by remote processing devices that are linked through a communications network. In such a distributed computing environment, program modules may be located in local and/or remote storage and/or memory systems.

As will be appreciated by one of skill in the art, the various embodiments of the present disclosure may be embodied as a method (including, for example, a computer-implemented process, a business process, and/or any other process), apparatus (including, for example, a system, machine, device, computer program product, and/or the like), or a combination of the foregoing. Accordingly, embodiments of the present disclosure may take the form of an entirely hardware embodiment, an entirely software embodiment (including firmware, middleware, microcode, hardware description languages, etc.), or an embodiment combining software and hardware aspects. Furthermore, embodiments of the present disclosure may take the form of a computer program product on a computer-readable medium or computer-readable storage medium, having computer-executable program code embodied in the medium, that define processes or methods described herein. A processor or processors may perform the necessary tasks defined by the computer-executable program code. Computer-executable program code for carrying out operations of embodiments of the present disclosure may be written in an object oriented, scripted or unscripted programming language such as Java, Perl, PHP, Visual Basic, Javascript, Python, Smalltalk, C++, or the like. However, the computer program code for carrying out operations of embodiments of the present disclosure may also be written in conventional procedural programming languages, such as the C programming language or similar programming languages. A code segment may represent a procedure, a function, a subprogram, a program, a routine, a subroutine, a module, an object, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, etc.

In the context of this document, a computer readable medium may be any medium that can contain, store, communicate, or transport the program for use by or in connection with the systems disclosed herein. The computer-executable program code may be transmitted using any appropriate medium, including but not limited to the Internet, optical fiber cable, radio frequency (RF) signals or other wireless signals, or other mediums. The computer readable medium may be, for example but is not limited to, an electronic, magnetic, optical, electromagnetic, infrared, or semiconductor system, apparatus, or device. More specific examples of suitable computer readable medium include, but are not limited to, an electrical connection having one or more wires or a tangible storage medium such as a portable computer diskette, a hard disk, a random access memory (RAM), a read-only memory (ROM), an erasable programmable read-only memory (EPROM or Flash memory), a compact disc read-only memory (CD-ROM), or other optical or magnetic storage device. Computer-readable media includes, but is not to be confused with, computer-readable storage medium, which is intended to cover all physical, non-transitory, or similar embodiments of computer-readable media.

Various embodiments of the present disclosure may be described herein with reference to flowchart illustrations and/or block diagrams of methods, apparatus (systems), and computer program products. It is understood that each block of the flowchart illustrations and/or block diagrams, and/or combinations of blocks in the flowchart illustrations and/or block diagrams, can be implemented by computer-executable program code portions. These computer-executable program code portions may be provided to a processor of a general purpose computer, special purpose computer, or other programmable data processing apparatus to produce a particular machine, such that the code portions, which execute via the processor of the computer or other programmable data processing apparatus, create mechanisms for implementing the functions/acts specified in the flowchart and/or block diagram block or blocks. Alternatively, computer program implemented steps or acts may be combined with operator or human implemented steps or acts in order to carry out an embodiment of the invention.

Additionally, although a flowchart or block diagram may illustrate a method as comprising sequential steps or a process as having a particular order of operations, many of the steps or operations in the flowchart(s) or block diagram(s) illustrated herein can be performed in parallel or concurrently, and the flowchart(s) or block diagram(s) should be read in the context of the various embodiments of the present disclosure. In addition, the order of the method steps or process operations illustrated in a flowchart or block diagram may be rearranged for some embodiments. Similarly, a method or process illustrated in a flow chart or block diagram could have additional steps or operations not included therein or fewer steps or operations than those shown. Moreover, a method step may correspond to a method, a function, a procedure, a subroutine, a subprogram, etc.

As used herein, the terms “substantially” or “generally” refer to the complete or nearly complete extent or degree of an action, characteristic, property, state, structure, item, or result. For example, an object that is “substantially” or “generally” enclosed would mean that the object is either completely enclosed or nearly completely enclosed. The exact allowable degree of deviation from absolute completeness may in some cases depend on the specific context. However, generally speaking, the nearness of completion will be so as to have generally the same overall result as if absolute and total completion were obtained. The use of “substantially” or “generally” is equally applicable when used in a negative connotation to refer to the complete or near complete lack of an action, characteristic, property, state, structure, item, or result. For example, an element, combination, embodiment, or composition that is “substantially free of” or “generally free of” an element may still actually contain such element as long as there is generally no significant effect thereof.

In the foregoing description various embodiments of the present disclosure have been presented for the purpose of illustration and description. They are not intended to be exhaustive or to limit the invention to the precise form disclosed. Obvious modifications or variations are possible in light of the above teachings. The various embodiments were chosen and described to provide the best illustration of the principals of the disclosure and their practical application, and to enable one of ordinary skill in the art to utilize the various embodiments with various modifications as are suited to the particular use contemplated. All such modifications and variations are within the scope of the present disclosure as determined by the appended claims when interpreted in accordance with the breadth they are fairly, legally, and equitably entitled.

We claim:

1. A method for providing a tailored drug recommendation for a patient, the method comprising:
receiving a DNA sample for the patient;
identifying a plurality of metabolizer genes for the patient, the metabolizer genes related to the patient's ability to metabolize a drug, the metabolizer genes indicating a metabolizer gene phenotype for the patient;
comparing a drug to the metabolizer gene phenotype;
based on the comparison, assigning a classification to the drug; and
providing a recommendation for the patient with respect to the drug, the recommendation based at least in part on the assigned classification of the drug.

2. The method of claim 1, further comprising:
identifying an event/response gene for the patient, the event/response gene related to the patient's compatibility with the drug, the event/response gene further indicating an event/response gene phenotype for the patient; and
comparing the drug to the event response gene phenotype.

3. The method of claim 2, wherein the classification is a metabolizer classification, the method further comprising:
based on the comparison of the drug to the event/response gene phenotype, assigning an event/response classification to the drug; and
assigning an overall drug classification to the drug, the overall drug classification based on the metabolizer classification and the event/response classification.

4. The method of claim 1, wherein comparing a drug to the metabolizer gene phenotype comprises:
calculating a metabolizer score for each metabolizer gene;
identifying one or more of the plurality of metabolizer genes capable of metabolizing the drug;
assigning a weight to the one or more metabolizer genes capable of metabolizing the drug;
multiplying the metabolizer score for each metabolizer gene capable of metabolizing the drug by its assigned weight to obtain a weighted metabolizer score; and
summing the weighted metabolizer scores to obtain a drug score for the drug.

5. The method of claim 4, wherein assigning a classification to the drug comprises comparing the drug score for the drug to a plurality of classifications.

6. The method of claim 1, wherein the metabolizer classification is one of: use as directed, use with caution, and use with great caution.

7. The method of claim 3, wherein the event/response classification is one of: use as directed, use with caution, and use with great caution.

8. The method of claim 3, wherein the overall drug classification is one of: use as directed, use with caution, and use with great caution.

9. The method of claim 3, wherein the overall drug classification is one of the metabolizer classification and the event/response classification, whichever signifies the most caution.

10. The method of claim 1, wherein the metabolizer phenotype comprises one of: poor metabolizer, poor to intermediate metabolizer, intermediate metabolizer, intermediate to normal metabolizer, normal metabolizer, normal to ultrarapid metabolizer, ultrarapid metabolizer, intermediate to ultrarapid metabolizer, poor to normal metabolizer, and poor to ultrarapid metabolizer.

11. The method of claim 1, wherein providing a recommendation comprises providing a report.

12. The method of claim 1, wherein the recommendation is provided to a medical professional.

13. The method of claim 1, wherein the recommendation is provided to the patient.

14. The method of claim 1, wherein providing a recommendation comprises providing an interactive recommendations terminal.

15. The method of claim 1, wherein identifying a plurality of metabolizer genes for the patient comprises identifying the alleles for each of the metabolizer genes.

16. The method of claim 4, wherein calculating a metabolizer score for each metabolizer gene comprises, for each metabolizer gene:
identifying the alleles for the gene;
assigning an allele score to each allele; and
summing the allele scores to obtain a metabolizer score.

17. A method for providing a tailored drug recommendation for a patient, the method comprising:
receiving DNA data for the patient;
identifying a genotype for the patient with respect to at least one of: a metabolizer gene related to the patient's ability to metabolize a drug and an event/response gene related to the patient's compatibility with the drug;
identifying a phenotype for the patient with respect to the at least one of a metabolizer gene and an event/response gene; and
providing a recommendation for the patient with respect to the drug.

18. The method of claim 17, wherein providing a recommendation comprises comparing the drug to the phenotype.

19. A system for providing a tailored drug recommendation for a patient, the system comprising:
a database storing data related to one or more drug-gene relationships; and
a controller configured to provide a tailored drug recommendation, the controller comprising:
a patient evaluation module configured to evaluate DNA data for the patient to identify one or more genes related to the patient's ability to metabolize a drug;
a drug evaluation module configured to compare the one or more identified genes with the drug; and
a recommendations module configured to provide one or more tailored drug recommendations for the patient.

20. The system of claim 19, further comprising a user interface providing the one or more tailored drug recommendations through an interactive recommendations terminal.
